SEQUENCE LISTING (4 sequences)

SEQ ID NO: 1 - DNA, 27 bp, artificial
  Artifical - Chemically synthesized.
atcgaattccggatcaggcggagaaga

SEQ ID NO: 2 - DNA, 27 bp, artificial
  Artifical - Chemically synthesized.
taaggatccctgacgcatgcccctatc

SEQ ID NO: 3 - DNA, 27 bp, artificial
  Artifical - Chemically synthesized.
cgcggatccgaatcctaatggacctgg

SEQ ID NO: 4 - DNA, 27 bp, artificial
  Artifical - Chemically synthesized.
taggaattctccaggctctcgcgcaag

(19) United States
(12) Patent Application Publication
(10) Pub. No.: US 2008/0254062 A1
(43) Pub. Date: Oct. 16, 2008

(54) USE OF AN AVIRULENT BORDETELLA MUTANT AS A LIVE VACCINE VECTOR

(75) Inventor: Eric Harvill, State College, PA (US)

Correspondence Address: MCKEE, VOORHEES & SEASE, P.L.C.;ATTN: PENNSYLVANIA STATE UNIVERSITY, 801 GRAND AVENUE, SUITE 3200, DES MOINES, IA, 50309-2721, US

(73) Assignee: THE PENN STATE RESEARCH FOUNDATION, University Park, PA (US)

(21) Appl. No.: 12/036,808

(22) Filed: Feb. 25, 2008

Related U.S. Application Data
(60) Provisional application No. 60/891,375, filed on Feb. 23, 2007.

(51) Int. Cl. A61K 39/00; C12N 1/20; A61P 37/00

(52) U.S. Cl. 424/200.1; 424/253.1; 424/254.1; 435/245

(57) ABSTRACT

The present invention pertains to Bordetella bacteria having a double mutation, a first mutation in a gene of the Type III secretion system and a second mutation in a gene of the adenylate cyclase toxin (CyaA) locus of the bacteria so that the mutations result in no Type III secretion system, a non-functional Type III secretion system, no CyaA protein, or a non-functional CyaA protein or a combination thereof. The Bordetella bacteria double mutant is attenuated while maintaining the efficacy of the bacteria to elicit an immune response. The present invention also pertains to vaccine compositions and methods for treating and immunizing a mammal against a disease caused by infection of Bordetella bacteria or a disease caused by a pathogen.

### CROSS-REFERENCE TO RELATED APPLICATIONS

This application claims priority under 35 U.S.C. § 119 of a provisional application Ser. No. 60/891,375 filed Feb. 23, 2007, which application is hereby incorporated by reference in its entirety.

### GRANT REFERENCE

This invention was developed with government support under USDA funding provided under the Hatch Act for project PEN03846, the United States Department of Agriculture 2002-35204-11684 and the National Institutes of Health 5-RO1-A1053075-02. The government has certain rights in this invention.

### BACKGROUND OF THE INVENTION

The lower respiratory tract has a well-developed immunological surveillance system which, during health, maintains this area as a sterile environment despite constant exposure to microorganisms. However, some microorganisms specialize in infecting the mammalian respiratory tract suggesting that they have evolved ways to modulate or avoid host defense mechanisms. One such microorganism is the bacteria of the Bordetella genus.

It is known that the Bordetella, more particularly Bordetella pertussis, Bordetella parapertussis and Bordetella bronchiseptica, are responsible for respiratory diseases in vertebrates, including but not limited to kennel cough, whooping cough, atrophic rhinitis and turbinate atrophy.

Since respiratory infections are a major source of morbidity and mortality, the development of vaccines that can protect against these infectious organisms is a priority. Historically, vaccination strategies have focused on the development of strong serum antibody titers as an indicator of efficacy, however, serum antibody titers do not always correlate with protection, particularly against mucosal pathogens. While parenteral vaccination against respiratory pathogens often protects against disease, it does not always prevent infection. Additionally, immunity induced by a bolus injection often wanes, leaving individuals susceptible to disease. In many cases, protective immunity generated in response to local mucosal infection is more effective and longer lasting than that generated in response to parenteral immunization (reviewed in (Moylett, E. H., and I. C. Hanson. 2003. 29. Immunization. J Allergy Clin Immunol 111:S754-765) and (Neutra, M. R., and P. A. Kozlowski. 2006. Mucosal vaccines: the promise and the challenge. Nat Rev Immunol 6:148-158).

The bordetellae efficiently and rapidly colonize ciliated respiratory epithelium and are able to persist within the host respiratory tract for several weeks. The mouse model provides an ideal system to study the potential use of live vaccines in a vigorous infection model in which both pathogen and host immunity can be experimentally manipulated. Attenuated strains of B. bronchiseptica have been used as live vaccines in a variety of domesticated mammals with limited data on safety and efficacy (reviewed in Stevenson, A., and M. Roberts. 2003. Use of Bordetella bronchiseptica and Bordetella pertussis as live vaccines and vectors for heterologous antigens. FEMS Immunol Med Microbiol 37:121-128). However, the molecular basis for attenuation is either unknown or unpublished. Since the genetic mutation in these strains has not been elucidated, the possibility of reversion to a more virulent form cannot be ruled out particularly with the wide variety of hosts, environments and exacerbating conditions and co-infections that may be encountered with their wide use. As a result, more recent studies have focused on the use of an attenuated strain of B. bronchiseptica, with a genetically defined mutation in the aroA gene, as live vaccines and as vectors for heterologous antigens (Bey, R. F., F. J. Shade, R. A. Goodnow, and R. C. Johnson. 1981. Intranasal vaccination of dogs with live avirulent Bordetella bronchiseptica: correlation of serum agglutination titer and the formation of secretory IgA with protection against experimentally induced infectious tracheobronchitis. Am J Vet Res 42:1130-1132; McArthur, J. D., N. P. West, J. N. Cole, H. Jungnitz, C. A. Guzman, J. Chin, P. R. Lehrbach, S. P. Djordjevic, and M. J. Walker. 2003. An aromatic amino acid auxotrophic mutant of Bordetella bronchiseptica is attenuated and immunogenic in a mouse model of infection. FEMS Microbiol Lett 221:7-16; Roberts, M., D. Maskell, P. Novotny, and G. Dougan. 1990. Construction and characterization in vivo of Bordetella pertussis aroA mutants. Infect Immun 58:732-739; Stevenson, A., and M. Roberts. 2004. Intranasal immunisation against tetanus with an attenuated Bordetella bronchiseptica vector expressing FrgC: improved immunogenicity using a Bvg-regulated promoter to express FrgC. Vaccine 22:4300-4305; Stevenson, A., and M. Roberts. 2002. Use of a rationally attenuated Bordetella bronchiseptica as a live mucosal vaccine and vector for heterologous antigens. Vaccine 20:2325-2335). Studies in dogs using current live attenuated B. bronchiseptica vaccines show mixed results. One study done by Jacobs et al showed partial protection (Jacobs, A. A., R. P. Theelen, R. Jaspers, L. J. Horspool, D. Sutton, J. G. Bergman, and G. Paul. 2005. Protection of dogs for 13 months against Bordetella bronchiseptica and canine parainfluenza virus with a modified live vaccine. Vet Rec 157:19-23), while other studies indicate that several live attenuated B. bronchiseptica vaccines are ineffectual and cause adverse effects (Edinboro, C. H., M. P. Ward, and L. T. Glickman. 2004. A placebo-controlled trial of two intranasal vaccines to prevent tracheobronchitis (kennel cough) in dogs entering a humane shelter. Prev Vet Med 62:89-99).

Since protection is often associated with immune responses to virulence factors, efforts have been focused on generating mutants with metabolic defects which alter the ability to survive in vivo but allow for expression of virulence factors (reviewed in (Raupach, B., and S. H. Kaufmann. 2001. Bacterial virulence, proinflammatory cytokines and host immunity: how to choose the appropriate Salmonella vaccine strain? Microbes Infect 3:1261-1269; Stevenson, A., and M. Roberts. 2003. Use of Bordetella bronchiseptica and Bordetella pertussis as live vaccines and vectors for heterologous antigens. FEMS Immunol Med Microbiol 37:121-128).

Many of the strains that are used in a live vaccine do not induce a sufficiently high level of immunity. Disadvantageously, many of the strains have unknown mutations leaving open the possibility of a reversion to wild type virulence.

The best studied B. bronchiseptica vaccine strains with defined mutations have a disruption in aroA, a gene which encodes a synthase crucial to the production of aromatic amino acids (Stevenson, A., and M. Roberts. 2002. Use of a rationally attenuated Bordetella bronchiseptica as a live mucosal vaccine and vector for heterologous antigens. Vaccine 20:2325-2335). The aroA mutant is considerably less efficient at colonizing the respiratory tract of mice as it is cleared by day 8 post inoculation as compared to its wild type parent which persists until at least day 28 post-inoculation (Stevenson, A., and M. Roberts. 2002. Use of a rationally attenuated Bordetella bronchiseptica as a live mucosal vaccine and vector for heterologous antigens. Vaccine 20:2325-2335). Although these mutants seem to generate protective immunity against their parental strains, the anti-B. bronchiseptica titers in mice infected with the mutant strain were 1/100 that of mice infected with wild type strain, suggesting that optimal antibody production requires efficient colonization (McArthur, J. D., N. P. West, J. N. Cole, H. Jungnitz, C. A. Guzman, J. Chin, P. R. Lehrbach, S. P. Djordjevic, and M. J. Walker. 2003. An aromatic amino acid auxotrophic mutant of Bordetella bronchiseptica is attenuated and immunogenic in a mouse model of infection. FEMS Microbiol Lett 221:7-16). Stevenson et al recently demonstrated the potential of using the aroA mutant to deliver a fragment of the tetanus toxoid (FrgC) in that both humoral and mucosal antibody responses to tetanus toxoid were measurable (Stevenson, A., and M. Roberts. 2004. Intranasal immunisation against tetanus with an attenuated Bordetella bronchiseptica vector expressing FrgC: improved immunogenicity using a Bvg-regulated promoter to express FrgC. Vaccine 22:4300-4305). However, this vaccine only protected a minority of the mice from challenge with lethal doses of toxin.

In summary, the current state of the art of the use of live Bordetella vaccines is that most of the live B. vaccines have no published data on vaccine efficacy in terms of protection from subsequent infection. Those with published efficacy, such as the aroA mutant, all have decreased efficacy compared to that generated by wild type strains. Thus, although many groups have attempted to create a strain that causes no disease but induces immunity as effective as a virulent strain, none have succeeded to date. In fact, some have argued that it is not possible to separate the ability to cause disease from the induction of effective protective immunity, in which case the safer a vaccine could be made the less effective it would be in preventing subsequent disease. Therefore, for these and other reasons there is a need for a Bordetella strain that could be safe in normal, and even in immunodeficient hosts, and yet retain the ability to induce protective immunity substantially the same as that induced by a virulent form of this bacterium.

### DETAILED DESCRIPTION OF THE FIGURES

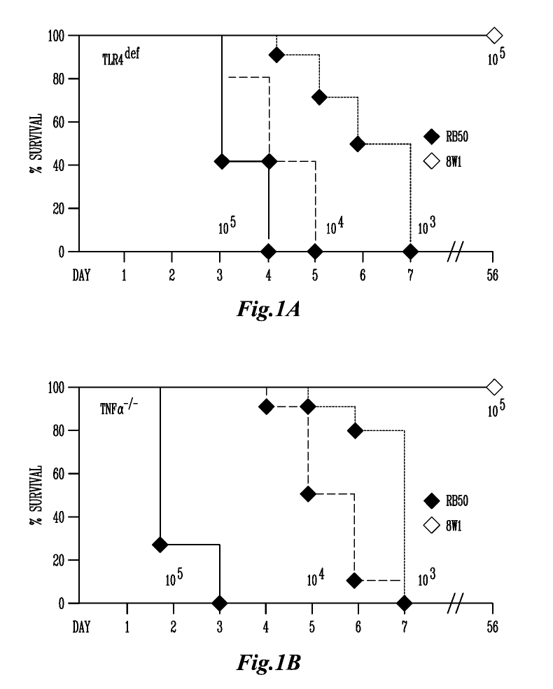

FIG. 1. Lethality of B. bronchiseptica strains in susceptible mice. Groups of 5 to 10 A) TLR4^{def} or B) TNFα^{-/-} mice were intranasally inoculated with approximately 5×10³, 5×10⁴ or 5×10⁵ CFU of RB50 or 5×10⁵ CFU of AVS in 50 μL as indicated.

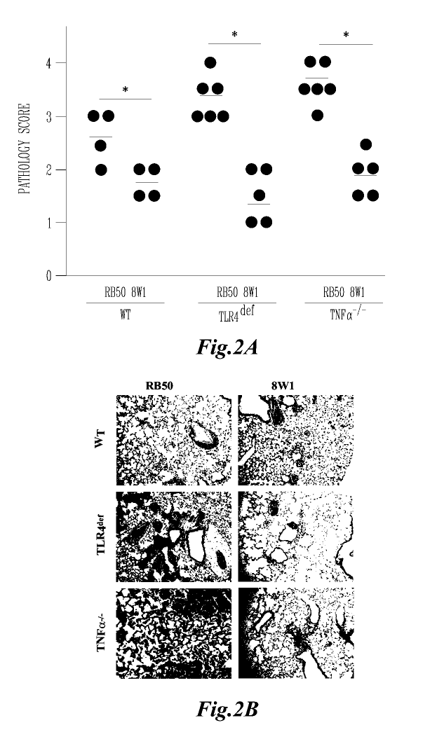

FIG. 2. Lung pathology in susceptible mouse strains. Groups of 4 to 6 WT, TLR4^{def} or TNFα^{-/-} mice were intranasally inoculated with approximately 5×10⁵ CFU of either RB50 or AVS in 50 μL as indicated. On day 3 post-inoculation the trachea and lungs were excised, inflated with 10% formaldehyde and then sectioned, stained and examined by a veterinary pathologist blinded to experimental treatment (M.J.K.). A) Pathology scores and B) lung histology pictures are shown. * indicates P value of <0.05. Error bars indicate standard error.

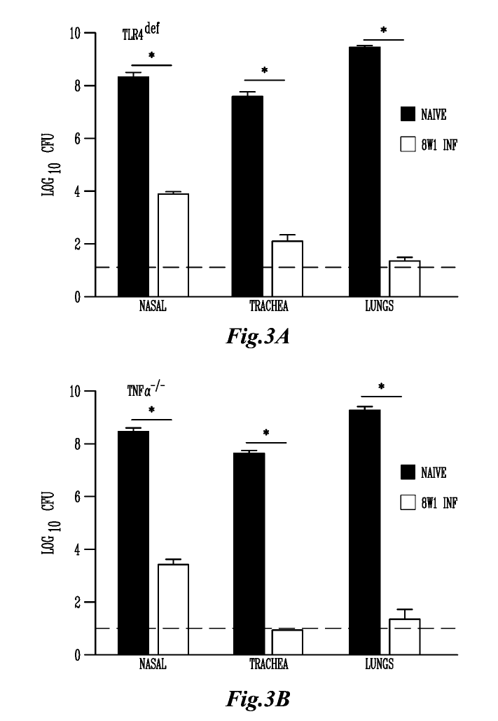

FIG. 3. AVS protection in susceptible mouse strains. Groups of 4 A) TLR4^{def} or B) TNFα^{-/-} mice were intranasally inoculated with approximately 5×10⁵ CFU of AVS in 50 μL. On day 49 post-inoculation, the mice were then challenged with approximately 5×10⁵ CFU of RB50 in 50 μL. On day 52, the mice were sacrificed and the number of RB50 CFU in the nasal cavity, trachea and lungs were measured. The dashed line indicates the limit of detection. * indicates P value of <0.05. Error bars indicate standard error.

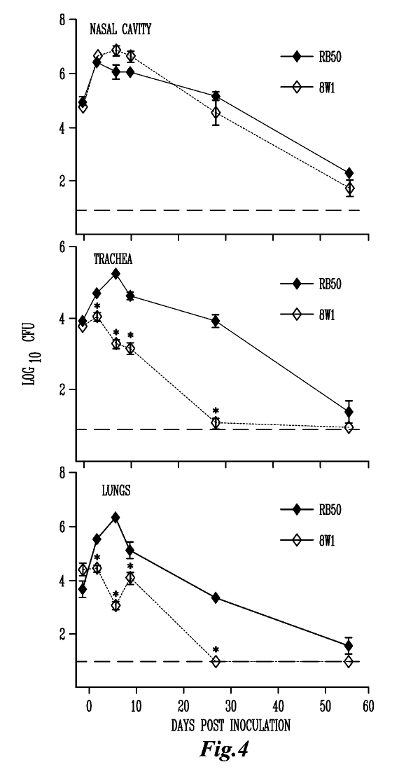

FIG. 4. AVS colonization in wild type mice. Groups of 4 to 6 WT mice were intranasally inoculated with approximately 5×10⁵ CFU of either RB50 or AVS in 50 μL as indicated. Bacterial numbers were measured in the nasal cavity, trachea and lungs on days 0, 3, 7, 10, 28 and 49 post-inoculation. The dashed line indicates the limit of detection. * indicates P value of <0.05. Error bars indicate standard error.

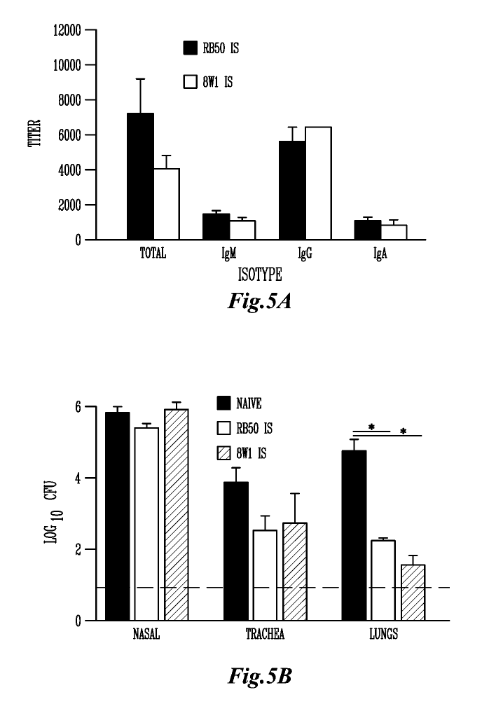

FIG. 5. AVS induced antibody response and its effect on RB50 colonization. A) Groups of 3 to 4 WT mice were intranasally inoculated with approximately 5×10⁵ CFU of either wild type RB50 or the AVS mutant in 50 μL as indicated. Serum was collected and pooled on day 49 post-inoculation and B. bronchiseptica-specific antibody titers were measured by ELISA. B) Groups of 4 WT mice were intraperitoneally injected with 200 μL of either naïve serum (NS), or immune serum (IS) raised against RB50 or AVS as indicated and intranasally inoculated with approximately 5×10⁵ CFU of RB50 in 50 μL. Bacterial numbers in the lungs, trachea and nasal cavity were measured on day 3 post-inoculation and transfer. The dashed line indicates the limit of detection. * indicates P value <0.05. Error bars indicate standard error.

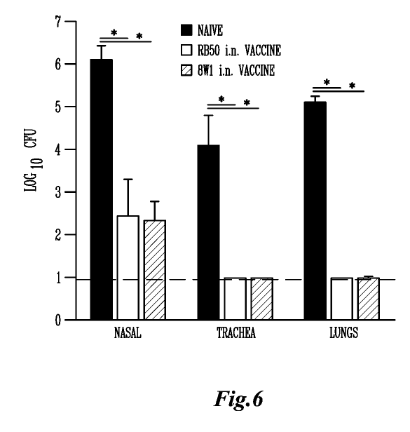

FIG. 6. Intranasal vaccination with AVS protects WT mice. Groups of 4 WT mice were intranasally vaccinated with approximately 100 CFU of either RB50 or AVS in a 5 μL volume. On day 49-post vaccination, the mice were intranasally inoculated with approximately 5×10⁵ CFU of RB50 in 50 μL and bacterial numbers were measured 3 days post-inoculation. The dashed line indicates the limit of detection. * indicates P value of <0.05. Error bars are standard error.

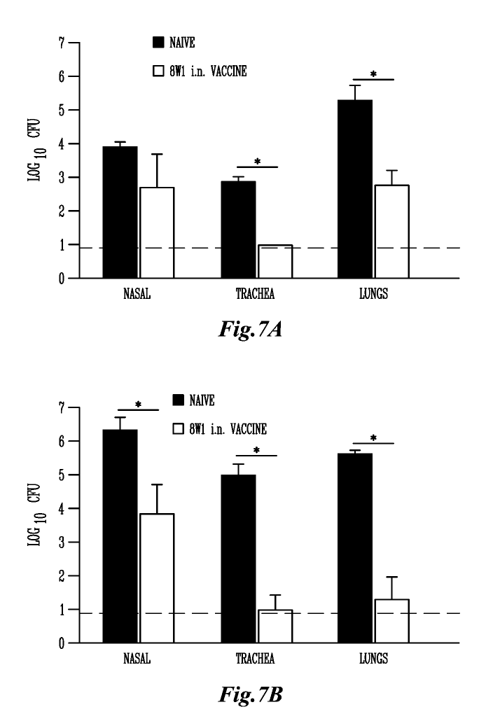

FIG. 7. Intranasal vaccination with AVS induces protective immunity against Bordetella pertussis and Bordetella parapertussis in the lower respiratory tract. WT mice were intranasally vaccinated with approximately 100 CFU of AVS in a 5 μL volume. On day 49-post vaccination, the mice were intranasally inoculated with approximately 5×10⁵ CFU of either A) B. pertussis or B) B. parapertussis in 50 μL as indicated. Bacterial numbers were measured 3 days post-inoculation. The dashed line indicates the limit of detection. * indicates P value of <0.05. Error bars indicate standard error.

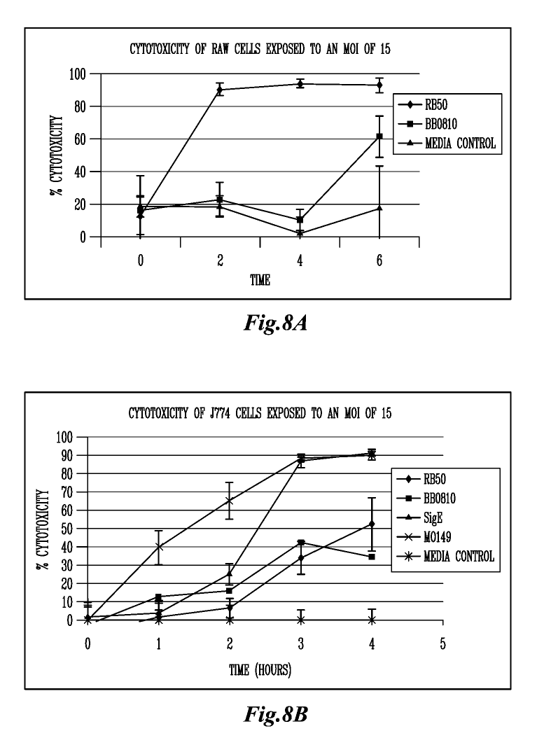

FIG. 8. Cytotoxicity of various bacterial strains for Raw (A) or J774 (B) cells in vitro.

### BRIEF SUMMARY OF THE INVENTION

Generally, it is the object of the present invention to provide Bordetella bacteria having at least one mutation in a gene of the Type III secretion system and at least one mutation in a gene of the adenylate cyclase toxin (cyaA) locus, e.g. adenylate cyclase toxin (cyaA), so that the corresponding proteins of the Type III secretion system and cyaA locus are not produced or are non-functional or a combination thereof. In one aspect, the mutation in the gene of the Type III secretion system results in the production of no Type III secretion system or a non-functional Type III secretion system. In a preferred embodiment, the mutations are deletions of part or all of the genes or the insertion of heterologous DNA-fragments or both. Advantageously, the defined mutations, unlike classically induced chemical mutations, prevent the reversion to a wild type virulence phenotype.

Because of their unexpected attenuated but immunogenic character in vivo, the bacteria are suitable as a basis for live attenuated vaccines. Bacteria having this double mutation when administered to a mammal have been found to be attenuated but able to induce protective immunity against Bordetella. According to the invention, the live attenuated double mutant Bordetella bacteria may be used in the preparation of live attenuated vaccine compositions. Therefore, it is an object of the present invention to provide vaccines comprising the double mutant Bordetella bacteria. In one aspect, the vaccine composition includes an adjuvant, a pharmaceutically acceptable carrier or both. Additionally, it is an object of the present invention to provide methods of immunizing a mammal against a disease caused by infection of Bordetella bacteria using vaccine compositions of the present invention. The method includes administering to a susceptible mammal an immunizing amount of a vaccine composition of the double mutant Bordetella bacteria. In a preferred embodiment, the vaccine composition is administered intranasally. The present invention also relates to preparing a Bordetella vaccine composition by mixing an immunizing amount of a vaccine composition of the double mutant Bordetella bacteria with a pharmaceutically acceptable carrier.

Provided herein in another aspect of the invention are methods for treating a disease caused by Bordetella infection in a mammal using double mutant Bordetella bacteria of the present invention. The method includes administering to a susceptible mammal an effective amount of double mutant Bordetella bacteria. The double mutant Bordetella bacteria and vaccine compositions thereof may elicit upon administration to a mammal a humoral immune response, cell-mediated immune response or both.

In another aspect, the invention includes a live attenuated vaccine composition for immunizing a mammal against a disease. The vaccine includes an immunizing amount of an avirulent double mutant bacteria that induces an immune response upon administration to a mammal. The vaccine may include a pharmaceutically acceptable carrier, an adjuvant or both.

The invention also includes a method of immunizing a mammal against a disease caused by a pathogen. In one aspect, the method includes administering to a susceptible mammal an immunizing amount of the double mutant Bordetella bacteria, where at least one gene of the Type III secretion system and a gene of adenylate cyclase toxin (CyaA) locus of the bacteria each comprise at least one mutation so that the Bordetella bacteria produce no Type III secretion system or a non-functional Type III secretion system and no CyaA protein or a non-functional CyaA protein. The double mutant Bordetella bacteria further include a heterologous gene encoding an antigen derived from the pathogen and a pharmaceutically acceptable carrier. In a preferred embodiment, the heterologous gene encodes an antigen derived from Leptospira canicola, Leptospira grippotyphosa, Leptospira hardjo, Leptospira ictero-haemorrhagiae, Leptospira pomona, Leptospira interrogans, Leptospira bratislava, canine distemper virus, canine adenovirus type 2, canine parainfluenza virus, canine parvovirus, rabies, herpes viruses, HIV, Erysipelothrix rhusiopathiae, Pasteurella, Pasteurella multocida, Ascaris, Oesophagostomum, pseudorabies virus, porcine parvovirus, pathogenic Escherichia coli, Bacillus anthracis, respiratory syncytial virus, Porcine Reproductive and Respiratory Syndrome Virus (PRRSV), swine influenza virus (SIV), porcine circovirus (PCV), Clostridium, Salmonella, Vibrio, Mycoplasma, Actinobacillus pleuropneumoniae, Haemophilus, rotavirus, transmissible gastroenteritis virus, Streptococcus sobrinus, Streptococcus mutans, and influenza.

Additionally, it is an object of the present invention to provide methods for treating a disease caused by a pathogen in a mammal. The method includes administering to a susceptible mammal an immunizing amount of a double mutant Bordetella bacteria, where at least one gene of the Type III secretion system and a gene of adenylate cyclase toxin (CyaA) locus of the bacteria each comprise at least one mutation so that the Bordetella bacteria produce no Type III secretion system or a non-functional Type III secretion system and no CyaA protein or a non-functional CyaA protein. The double mutant Bordetella bacteria further include a heterologous gene encoding an antigen derived from the pathogen and a pharmaceutically acceptable carrier. In a preferred embodiment, the heterologous gene encodes an antigen derived from Leptospira canicola, Leptospira grippotyphosa, Leptospira hardjo, Leptospira ictero-haemorrhagiae, Leptospira pomona, Leptospira interrogans, Leptospira bratislava, canine distemper virus, canine adenovirus type 2, canine parainfluenza virus, canine parvovirus, rabies, herpes viruses, HIV, Erysipelothrix rhusiopathiae, Pasteurella, Pasteurella multocida, Ascaris, Oesophagostomum, pseudorabies virus, porcine parvovirus, pathogenic Escherichia coli, Bacillus anthracis, respiratory syncytial virus, Porcine Reproductive and Respiratory Syndrome Virus (PRRSV), swine influenza virus (SIV), porcine circovirus (PCV), Clostridium, Salmonella, Vibrio, Mycoplasma, Actinobacillus pleuropneumoniae, Haemophilus, rotavirus, transmissible gastroenteritis virus, Streptococcus sobrinus, Streptococcus mutans, and influenza.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### DETAILED DESCRIPTION OF THE INVENTION

The present invention now will be described more fully hereinafter with reference to the accompanying examples, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the invention set forth herein will come to mind to one skilled in the art to which this invention pertains, having the benefit of the teachings presented in the descriptions and the drawings herein. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation. The articles “a” and “an” are used herein to refer to one or more than one (i.e., to at least one) of the grammatical object of the article. By way of example, “an element” means one or more than one element. As used herein, “bacteria” and “bacterium” are used interchangeably. As used herein, “vaccine composition” and “vaccine” are used interchangeably. As used herein, “Type III secretion system”, “Type three secretion system”, and TTSS are used interchangeably.

In one embodiment, the present invention includes live attenuated Bordetella bacteria that have mutations in a gene of the Type III secretion system and in a gene of the cyaA locus so that the bacteria produce no corresponding Type III secretion system protein or a corresponding non-functional type III secretion system protein and no corresponding protein of the cyaA locus, e.g. CyaA, or a non-functional CyaA protein or a combination thereof for use in a vaccine. Also, included are mutant Bordetella bacteria obtained by knocking down or inhibiting genes in addition to or other than cyaA locus genes and/or Type III secretion genes, for example, those upstream or downstream of cyaA and Type III secretion genes in a regulatory cascade that result in an avirulent mutant that is immunogenic.

The products of the Type III secretion system are a series of proteins that regulate the export of virulence factors to host cells. Adenylate cyclase toxin (CyaA) is a bacterial endotoxin produced by Bordetella that converts cellular ATP to cAMP in eukaryotic cells to cytotoxic levels and has been shown to play an important role during the early phase of lung colonization by the Bordetella bacteria. (Harvill, E. T., Cotter, P. A., Yuk, M. H., Miller, J. F.: Probing the function of Bordetella bronchiseptica adenylate cyclase toxin by manipulating host immunity. Infect. Immun. 67, 1493-1500 (1999).) Bordetella bacteria lacking these two mechanisms of a functional type III secretion system and a functional adenylate cyclase toxin (CyaA) protein are avirulent, in that they do not induce lung pathology in normal animals and do not overcome and kill various immunodeficient animals, as the normal (parental) Bordetella does. However, despite expectations that bacteria having multiple mutations in its genome would die, not thrive or be unable to replicate, the present invention demonstrates that Bordetella bacteria lacking these two mechanisms induces an antibody response that is as strong and protective immunity that is as effective as that induced by the wild type strain (parental strain) of Bordetella, contrary to the dogma and prior published reports with other strains.

The double mutation provides an effective means to attenuate the Bordetella to provide a safe and efficacious vaccine composition. The vaccines, live attenuated Bordetella bacteria double mutants, and methods of the present invention may be used to immunize against and/or to treat a disease caused by infection of Bordetella bacteria. Additionally, methods of preparing vaccines useful for immunizing against and/or treating Bordetella infection are also provided. In an embodiment, the invention relates to live attenuated bacteria of the genus of Bordetella for use in a vaccine. As used herein, Bordetella includes any bacteria belonging to the genus of Bordetella, for example, members of the species Bordetella ansorpii (B. ansorpii), Bordetella avium (B. avium), Bordetella pertussis (B. pertussis), Bordetella parapertussis (B. parapertussis), Bordetella bronchiseptica (B. bronchiseptica), Bordetella avium (B. avium), Bordetella holmesii (B. holmesii), Bordetella petrii (B. petrii), Bordetella trematum (B. trematum), Bordetella hinzii (B. hinzii) and the like.

In a more preferred form of the invention, the live attenuated bacteria according to the invention include but are not limited to the 8W1 (AVS) strain and other strains having no Type III Secretion System or a non-functional Type III Secretion System and no Adenylate Cyclase Toxin (CyaA) protein or a non-functional CyaA protein, or a combination thereof. See, for example, Yuk, M. H., Harvill, E. T., Miller J. F. The BvgAS Virulence Control System Regulates Type III Secretion in Bordetella Bronchiseptica. Mol. Microbiol. 1998 June; 28(5):945-59). As used herein, the term 8W1 and AVS are used interchangeably.

### A. Type III Secretion System

In one embodiment, the present invention includes live attenuated Bordetella bacteria having a mutation in one or more genes of the Type III secretion system, including any mutation that diminishes, abolishes or otherwise alters the effectiveness of the corresponding gene product (protein) so that the protein is not expressed or is non-functional in performing any of the functions it carries in vivo. A “non-functional” protein means that the gene encoding the protein has a mutation compared to a corresponding wild type gene such that the mutation inhibits or reduces expression and/or biological activity of the encoded gene product (protein). A functional protein is understood to be a protein having the regulating characteristics of the wild-type protein.

Exemplary mutations in genes of the Type III secretion system include (a) a mutation in a “core” protein that decreases or abolishes the ability of the Type III secretion system to secrete proteins or to translocate effectors into host cells or host cell membranes, (b) a mutation deleting or modifying an effector gene, such that a gene product is not produced or is non-functional, or (c) a mutation to other components of the system, chaperones for example, which are necessary for the delivery of the effectors in the wild type bacteria. Regulatory elements, transcription factors and other components used by the wild type bacteria may also be altered in such a manner that the transcription, translation and/or processing of a component or components of the system is altered. A Type III secretion system protein that is defective in at least one of its functions is considered to be a non-functional Type III secretion system protein. Type III secretion system functions include directing the secretion and translocation of a variety of proteins that cause species-specific pathogenesis phenotypes.

In one aspect, the mutation in a gene of the Type III secretion system results in no Type III secretion system or a non-functional Type III secretion system. The absence of a Type III secretion system includes a Type III secretion system that has no capacity to secrete BopN, BopD and/or other molecules secreted by the system, no capacity to kill cells in vitro, no capacity to induce TTSS-associated pathology, no capacity to induce lethal disease in mice or any combination of these. A “non-functional” Type III secretion system includes a Type III secretion system that has decreased capacity to secrete BopN, BopD and/or other molecules secreted by the system, a decreased capacity to kill cells in vitro, a decreased capacity to induce TTSS-associated pathology, a decreased capacity to induce lethal disease in mice or any combination of these. Levels of secreted BopN, BopD and/or other molecules secreted by the system may be determined, for example, by using ELISAs or other well-known techniques. A decreased level of BopN or BopD secreted by a bacteria mutant suspected of having no Type III secretion system or a non-functional Type III secretion system as compared to the level of BopN or BopD secreted by a wild type (parental) Bordetella with a wild type Type III secretion system indicates that the bacteria have no Type III secretion system or a non-functional Type III secretion system. The difference in levels may be statistically significant. A decreased number or percentage of killed cells in vitro by a bacteria mutant suspected of having no Type III secretion system or a non-functional Type III secretion system as compared to a wild type (parental) Bordetella with a wild type Type III secretion system indicates that the bacteria have no Type III secretion system or a non-functional Type III secretion system. One technique for detecting bacteria that have no Type III secretion system or a non-functional Type III secretion system is by cytotoxicity assays. See Harvill, E. T., P. A. Cotter, et al. (1999). “Probing the function of Bordetella bronchiseptica adenylate cyclase toxin by manipulating host immunity.” Infect Immun 67(3): 1493-500, herein incorporated by reference in its entirety. As an example, mammalian cells such as HeLa, MLE or 293T cells may be incubated with a bacteria mutant suspected of having no Type III secretion system or a non-functional Type III secretion system at a particular MOI, e.g. 100, for a certain period of time, such as 3 hours, and the cytoxicity of the bacteria on the cells measured as compared to a control, such as a parental wild type strain. See, for example, A Genome-Wide Screen Identifies A Bordetella Type III Secretion Effector and Candidate Effectors In Other Species. Molecular Microbiology (2005). 58(1):267-279, herein incorporated by reference in its entirety. The cytoxicity may be measured in any number of ways. It can be measured directly in terms of the number or percentage of killed mammalian cells or indirectly in the amount of lactate dehydrogenase (LDH) released by the mammalian cells. The bacteria mutant suspected of having no Type III secretion system or a non-functional Type III secretion system may have less than 40%, 35%, 30%, 25%, 20%, 15%, 10%, 5% or 1% cytotoxicity (as measured in the percentage of killed mammalian cells). A decrease in the level of LDH released by the mammalian cells incubated with the bacteria mutant suspected of having no Type III secretion system or a non-functional Type III secretion system as compared to the level of LDH released by the mammalian cells incubated with a control, e.g. a wild type (parental) Bordetella with a wild type Type III secretion system indicates that the candidate bacteria have no Type III secretion system or a non-functional Type III secretion system. The capacity to induce TTSS-associated pathology by a bacteria mutant suspected of having no Type III secretion system or a non-functional Type III secretion system may be determined by any suitable method including by determining the pathology in the lungs of a host administered the candidate bacteria as compared to a control, e.g. a wild type (parental) Bordetella. For example, candidate bacteria that have decreased numbers of lesions, decreased size of lesions or both than that observed with wild type (parental) strains with a wild Type III secretion system indicates that the candidate bacteria have no Type III secretion system or a non-functional Type III secretion system. Thus, Bordetella bacteria having one or more mutations in a gene of the Type III secretion system that result in no Type III secretion system or a non-functional Type III secretion system can be readily identified.

The mutation in the gene of the Type III secretion system may be naturally occurring, arise spontaneously, be induced, or be genetically engineered. In a preferred embodiment, the mutation is affected by deletion of part or all of the gene in the type III secretion system to hinder a spontaneous reversion in the gene to effect virulence of the bacteria. For example, the entire coding region of the gene can be deleted, leaving only the start and stop codons, so that transcriptionally linked genes are unaffected. Deletion of the entire coding region eliminates the possibility of a reversion mutation restoring activity. In one aspect, the double mutant Bordetella bacteria have a “stable” mutation in at least one of the genes encoding for a protein of the Type III secretion system or of the cyaA locus or both. As used herein, a “stable” mutation is one that is created by allelic exchange that does not leave any remnant DNA that might facilitate further mutations, such as insertion sequences, transposons or duplicated regions. A gene with a “stable” mutation should have no higher frequency of subsequent mutation than the original gene, or most other genes in the genome of the organism.

Spontaneous mutants, such as the majority of current live vaccine strains, contain only a small mutation inactivating a gene. These genes can obtain a ‘reversion’ mutation which can turn the gene back on, and render the strain virulent again. Another advantage of genetically engineering a mutation in the gene is that it provides a clear identification of the Bordetella mutant so that the Bordetella mutant can be distinguished from any others, for example, by polymerase chain reaction (PCR) and sequencing of the regions containing the mutations.

The genes of the Type III secretion system in B. bronchiseptica and other species, and their nucleotide sequences have been previously described. See WO99/59630 (PCT US99/10690, Comparative analysis of the genome sequences of Bordetella pertussis, Bordetella parapertussis and Bordetella bronchiseptica. Nat. Genet. 2003 September; 35(1):32-40. Epub 2003 Aug. 10, herein incorporated by reference in its entirety. The bacteria may have a mutation in one or more of the following genes: bscV, bcr3, bopN, bsp22, bcrH1, bopD, bopB, bcrH2, bcr4, bscl, bscj, bscK, bscL, bscN or bscO genes. The mutation may be in a “core” gene, or in an “effector” gene.

The bacteria may have a mutation in a bscN gene. In one aspect, the mutation in the bscN gene is as described herein in Example 11. In one aspect, the mutation in the bscN gene is as described in Yuk et al., “The BvgAS virulence control system regulates Type III secretion in Bordetella bronchiseptica”, Mol Microbiol 28:945-959 (1998), see also Example 15 described herein. The bacteria may include Type III secretion system mutants of Bordetella including but not limited to those having mutations, for example, deletions in bvgS, bscN, bsp22, bopD, btrS, btrS/pbtrS, btrU, btrW and btrV. See Akerley et al., “The bvgAS locus negatively controls motility and synthesis of flagella in Bordetella bronchiseptica”, J Bacteriol 174:980-990 (1992); Yuk et al., “The BvgAS virulence control system regulates Type III secretion in Bordetella bronchiseptica”, Mol Microbiol 28:945-959 (1998); Yuk et al., “Modulation of host immune responses, induction of apoptosis and inhibition of NF-kappaB activation by the Bordetella type III secretion system”, Mol Microbiol 35:991-1004 (2000); Mattoo et al., “Regulation of type III secretion in Bordetella”, Mol Microbiol 52:1201-1214 (2004); B. A. Medhekar et al., manuscript in preparation; Edwards et al., “Improved allelic exchange vectors and their use to analyze 987P fimbria gene expression”, Gene 207:149-157 (1998)); Kovach et al., “Four new derivatives of the broad-host-range cloning vector pBBR1MCS, carrying different antibiotic-resistance cassettes”, Gene 166:175-176 (1995), Harvill, E. T., P. A. Cotter, et al. (1999). “Probing the function of Bordetella bronchiseptica adenylate cyclase toxin by manipulating host immunity.” Infect Immun 67(3): 1493-500, herein incorporated by reference in its entirety.

Previous work involving Type III Bordetella mutants demonstrated that they were hypervirulent in SCID-Beige mice and administration resulted in more rapid death of these animals than observed with the wild type strain (Modulation of Host Immune Responses, Induction of Apoptosis and Inhibition of NF-kappaB Activation by the Bordetella Type III Secretion System. Mol. Microbiol. 2000 March; 35(5):991-1004).

### B. Adenylate Cyclase Toxin (CyaA)

In one embodiment, the present invention includes live attenuated Bordetella bacteria having a mutation in one or more genes of the cyaA locus, including any mutation that diminishes, abolishes or otherwise alters the effectiveness of the CyaA protein in performing any of the functions it carries in vivo, for example, the conversion of cellular ATP to cAMP in cells. Exemplary genes of the cyaA locus include cyaA, cyaB, cyaC, and cyaD. Exemplary mutations include (a) a mutation in a regulatory element of a gene of the cyaA locus, (b) a mutation in an intron or in an exon that encodes a protein of the cyaA locus, and (c) a mutation deleting or modifying the gene of the cyaA locus, such that a gene product is not produced or is non-functional, or otherwise attenuated. Exemplary mutations in cyaA include (a) a mutation in a regulatory element of the cyaA gene, (b) a mutation in an intron or in an exon that encodes an enzymatic domain of the CyaA protein, such as an AC or hemolysin (HLY) domain that decreases or abolishes the ability of the CyaA to convert cellular ATP to cAMP in host cells, and (c) a mutation deleting or modifying the cyaA gene, such that a gene product is not produced or is non-functional, or otherwise attenuated. CyaA protein that is defective in at least one of its functions is considered to be a non-functional CyaA protein. CyaA protein functions include the conversion of cellular ATP to cAMP.

In one aspect, the mutation in a gene of the cyaA locus results in the production of a corresponding non-functional protein encoded by the gene of the cyaA locus or no corresponding protein encoded by the gene of the cyaA locus. Without wishing to be bound by this theory, it is believed that a mutation in a gene of the cyaA locus results in producing a non-functional cyaA protein or no cyaA protein.

Bordetella bacteria having no CyaA protein or a non-functional CyaA protein, e.g. caused by mutations in CyaA gene, can easily be selected because of their phenotype, for example, their decreased ability to produce cAMP or colonize the respiratory tract in vivo as compared to a control. One technique for detecting bacteria that have no CyaA protein or a non-functional CyaA protein is by hemolysis assays. See, for example, Harvill, E. T., P. A. Cotter, et al. (1999). “Probing the function of Bordetella bronchiseptica adenylate cyclase toxin by manipulating host immunity.” Infect Immun 67(3): 1493-500, herein incorporated by reference in its entirety. Another technique for detecting bacteria that have no CyaA protein or a non-functional CyaA protein is by pathology assays. For example, a bacteria mutant suspected of having no CyaA protein or a non-functional CyaA protein may be identified by determining the pathology in the lungs of a host administered the candidate bacteria as compared to a control, e.g. a wild type (parental) Bordetella. For example, candidate bacteria that have decreased numbers of lesions, decreased size of lesions or both than that observed with wild type (parental) strains with a wild cyaA protein indicates that the candidate bacteria have no cyaA protein or a non-functional cyaA protein.

The mutation in one of the genes of the cyaA locus may be naturally occurring, arise spontaneously, be induced, or be genetically engineered. In a preferred embodiment, the mutation is affected by deletion of part or all of a gene of the cyaA locus, e.g. cyaA, to hinder a return to wild type phenotype. For example, the entire coding region of the gene can be deleted, leaving only the start and stop codons, so that transcriptionally linked genes are unaffected. Deletion of the entire coding region eliminates the possibility of a reversion mutation restoring activity. Spontaneous mutants, such as the majority of current live vaccine strains, are less desirable in that they may contain only a small mutation, generally a point mutation, inactivating a gene but leaving most of the gene present within the genome, and therefore allow the possibility that a small mutation may restore a functional gene. Another advantage of genetically engineering a mutation in the gene is that it provides a clear identification of the Bordetella mutant so that the Bordetella strain can be distinguished from any others, for example, by polymerase chain reaction (PCR) and sequencing of the regions containing the mutations.

The genes of the cyaA locus, e.g. adenylate cyclase toxin, and their nucleotide sequences in Bordetella bronchiseptica and other species have been previously described. See Comparative analysis of the genome sequences of Bordetella pertussis, Bordetella parapertussis and Bordetella bronchiseptica. Nat. Genet. 2003 September; 35(1):32-40. Epub 2003 Aug. 10, herein incorporated by reference in its entirety.

In one aspect, the bacteria have a mutation in the cyaC gene as described in “Characterization of adenylate cyclase toxin from a mutant of Bordetella pertussis defective in the activator gene, cyaC.” J Biol Chem 268(11): 7842-8, herein incorporated by reference in its entirety.

In one aspect, the bacteria have a mutation in the cyaA gene as described in Harvill, E. T., P. A. Cotter, et al. (1999). “Probing the function of Bordetella bronchiseptica adenylate cyclase toxin by manipulating host immunity.” Infect Immun 67(3): 1493-500, herein incorporated by reference in its entirety. In one aspect, the bacteria have a mutation in the cyaA gene as described in Yuk et al. The BvgAS Virulence Control System Regulates Type III Secretion in Bordetella Bronchiseptica. Mol. Microbiol. 1998 June; 28(5):945-59), herein incorporated by reference in its entirety.

Mutants in cyaA and cyaC have been shown to fail to produce functional Adenylate Cyclase Toxin and to have defects in their ability to grow rapidly in numbers and induce pathology in the respiratory tracts of mice. Characterization of adenylate cyclase toxin from a mutant of Bordetella pertussis defective in the activator gene, cyaC. J Biol Chem 268(11): 7842-8; Harvill, E. T., P. A. Cotter, et al. (1999). “Probing the function of Bordetella bronchiseptica adenylate cyclase toxin by manipulating host immunity.” Infect Immun 67(3): 1493-500, herein incorporated by reference in its entirety.

### C. Double Mutant Bordetella Bacteria

As described herein, the live attenuated Bordetella bacteria have a mutation in one or more genes of the Type III secretion system and in one or more genes of the cyaA locus, including any mutation that diminishes, abolishes or otherwise alters the effectiveness of the corresponding gene product (protein) so that the proteins are not produced or are non-functional in performing any of the functions it carries in vivo. In one aspect, the live attenuated double mutant bacteria have mutations in one or more genes of the Type III secretion system and in one or mores genes of the cyaA locus resulting in no Type III secretion system or a non-functional Type III secretion system and no corresponding protein encoded by the gene of the CyaA locus or a non-functional CyaA protein or combinations thereof. The double mutants may be used in a vaccine.

In one aspect, the live attenuated double mutant Bordetella bacteria have at least one mutation in one or more of the genes of the Type III secretion system including but not limited to bscV, bcr3, bopN, bsp22, bcrH1, bopD, bopB, bcrH2, bcr4, bscl, bscj, bscK, bscL, bscN, bteA, and bscO and in one or more the genes of the cyaA locus, including but not limited to cyaA, cyaB, cyaC, and cya D.

In one aspect, the bacteria have a mutation in the cyaC gene as described in “Characterization of adenylate cyclase toxin from a mutant of Bordetella pertussis defective in the activator gene, cyaC.” J Biol Chem 268(11): 7842-8, herein incorporated by reference in its entirety.

In one aspect, the bacteria have a mutation in the cyaA gene as described in Harvill, E. T., P. A. Cotter, et al. (1999). “Probing the function of Bordetella bronchiseptica adenylate cyclase toxin by manipulating host immunity.” Infect Immun 67(3): 1493-500, herein incorporated by reference in its entirety. Briefly, the region between the two consecutive ApaLI fragments, which encode the central 1,580 codons of CyaA within the cyaA gene of AVS, was deleted. In one aspect, the bacteria have a mutation in the cyaA gene as described in Yuk et al. The BvgAS Virulence Control System Regulates Type III Secretion in Bordetella Bronchiseptica. Mol. Microbiol. 1998 June; 28(5):945-59), herein incorporated by reference in its entirety. In one aspect, the bacteria lacks all of the cyaA gene except the first several codons and the last codons. These are maintained in-frame so that the ribosome will still start and stop at the appropriate positions on the messenger RNA, so that the translation of downstream genes are not affected. In another aspect, the bacteria lacks the entire cyaA gene, including start and stop codons, to eliminate concerns about possible effects of remnant small fragments of the gene.

The bacteria may have a mutation in the bscN gene. In one aspect, the bacteria have a mutation in the bscN gene as described herein in Example 11. In one aspect, the bacteria have a mutation in the bscN gene as described in Yuk et al., “The BvgAS virulence control system regulates type III secretion in Bordetella bronchiseptica”, Mol Microbiol 28:945-959 (1998), see also Example 15. In one aspect, the central region of the bscN gene in AVS was deleted, from codon 170 to codon 262. In one aspect, the bacteria have a mutation in the bscN gene as described in Harvill, E. T., P. A. Cotter, et al. (1999). “Probing the function of Bordetella bronchiseptica adenylate cyclase toxin by manipulating host immunity.” Infect Immun 67(3): 1493-500, herein incorporated by reference in its entirety. In one aspect, the bacteria have a deletion from codon 4 to 653 of the gene bteA.

In one aspect, the bacteria lacks all of a gene of the Type III secretion system except the first several codons and the last codons. These are maintained in-frame so that the ribosome will still start and stop at the appropriate positions on the messenger RNA, so that the translation of downstream genes are not affected. In another aspect, the bacteria lacks one or more entire genes the Type III secretion system, including start and stop codons, to eliminate concerns about possible effects of remnant small fragments of the gene.

The bacteria may include known Type III secretion system mutants of Bordetella including but not limited to those having mutations, for example, deletions in one or more of the following genes: bvgS, bscN, bsp22, bopD, btrS, btrS/pbtrS, btrU, btrW and btrV. See Akerley et al., “The bvgAS locus negatively controls motility and synthesis of flagella in Bordetella bronchiseptica”, J Bacteriol 174:980-990 (1992); Yuk et al., “The BvgAS virulence control system regulates type III secretion in Bordetella bronchiseptica”, Mol Microbiol 28:945-959 (1998); Yuk et al., “Modulation of host immune responses, induction of apoptosis and inhibition of NF-kappaB activation by the Bordetella type III secretion system”, Mol Microbiol 35:991-1004 (2000); Mattoo et al., “Regulation of type III secretion in Bordetella”, Mol Microbiol 52:1201-1214 (2004); B. A. Medhekar et al., manuscript in preparation; Edwards et al., “Improved allelic exchange vectors and their use to analyze 987P fimbria gene expression”, Gene 207:149-157 (1998)); Kovach et al., “Four new derivatives of the broad-host-range cloning vector pBBR1MCS, carrying different antibiotic-resistance cassettes”, Gene 166:175-176 (1995), Harvill, E. T., P. A. Cotter, et al. (1999). “Probing the function of Bordetella bronchiseptica adenylate cyclase toxin by manipulating host immunity.” Infect Immun 67(3): 1493-500, herein incorporated by reference in its entirety.

In one aspect, the double mutant has at least one mutation in the cyaA gene and in the bscN gene of the Type III secretion system of Bordetella species of Bordetella ansorpii, Bordetella avium, Bordetella pertussis, Bordetella parapertussis, Bordetella bronchiseptica, Bordetella avium, Bordetella holmesii, Bordetella petrii, Bordetella trematum, or Bordetella hinzii. In one aspect, the bacteria have a mutation in the cyaA gene and the bscN gene as described in Harvill, E. T., P. A. Cotter, et al. (1999). “Probing the function of Bordetella bronchiseptica adenylate cyclase toxin by manipulating host immunity.” Infect Immun 67(3): 1493-500, herein incorporated by reference in its entirety. In one aspect, the bacteria have a mutation in the cyaA gene and the bscN gene as described in Yuk et al. The BvgAS Virulence Control System Regulates Type III Secretion in Bordetella Bronchiseptica. Mol. Microbiol. 1998 June; 28(5):945-59), herein incorporated by reference in its entirety. In one aspect, the double mutant is the AVS (8W1) strain. Advantageously, the entire genome of the 8W1 Bordetella bronchiseptica has been sequenced by the Sanger Center. See Comparative analysis of the genome sequences of Bordetella pertussis, Bordetella parapertussis and Bordetella bronchiseptica. Nat Genet. 2003 September; 35(1):32-40. Epub 2003 Aug. 10, herein incorporated by reference in its entirety.

Previous work involving double mutant Bordetella bacteria containing a deletion in the bscN and the cyaA genes demonstrated that they were cytotoxic in vitro. Contrary to expectations, the live attenuated Bordetella double mutants of the present invention show decreased pathology but an increase in the immune response in vivo.

### D. Additional Mutations

In one embodiment, the present invention includes Bordetella bacteria that have in addition to a mutation in a gene encoding a protein of the Type III secretion system and in a gene of the cyaA locus, e.g. the cyaA gene, a mutation in at least one additional gene, for example, a gene that encodes a regulator of one or more virulence genes. The mutation in the additional gene does not necessarily have to be within the gene to disrupt the function. For example, a mutation in an upstream regulatory region of the gene may also disrupt gene expression, leading to attenuation. Mutations in an intergenic region may also be sufficient to disrupt gene function so that the gene's function is decreased or abolished. Exemplary genes that regulate one or more virulence genes include Sigma Factor SigE and those in the Type Six Secretion System, for example, BB0810. As appreciated by one skilled in the art, deletions of part or all of the Sigma Factor SigE gene or genes of Six Secretion System may be accomplished by any suitable method. See, for example, Examples 12-13.

In another aspect of the invention, the Bordetella double mutants for use in the vaccines and methods of the present invention are engineered so that the mutants have diminished ability to grow outside of the host, such as an auxotroph, or to transmit between hosts, for example, including but not limited to genes involving motility or nutrient utilization, such as flaA.

### II. How to Make Mutants

Bordetella bacteria having a double mutation, i.e. a mutation in a gene of the Type III secretion system and in a gene of the cyaA locus, e.g. the cyaA gene, may be identified or created using standard techniques, for example, chemical induction or recombinant DNA technology or combinations thereof. One possible way of mutating a gene encoding a protein of the Type III secretion system or a gene of the cyaA locus, e.g. cyaA gene, is by means of classical methods such as the treatment of Bordetella bacteria with mutagenic agents such as base analogues, treatment with ultraviolet light or temperature treatment (Anderson, P. 1995. Mutagenesis, p 31 58 in Methods in Cell Biology 48. H. F. Epstein and D.C. Shakes (Eds)).

The exact nature of the mutation caused by classical mutation techniques is usually unknown. This can be a point mutation which may eventually revert to wild-type. In some cases, it may be desirable to make the Bordetella double mutants using transposon mutagenesis or recombinant DNA techniques. Mutation by transposon mutagenesis is a mutagenesis-technique well-known in the art that can be used to create a mutation at a localized site in the chromosome.

It is preferred that a mutation is introduced at a predetermined site using recombinant DNA-technology. Recombinant DNA techniques relate to cloning of the gene, modification of the gene sequence by site-directed mutagenesis, restriction enzyme digestion followed by re-ligation or PCR-approaches and to subsequent replacement of the wild type gene with the mutant gene (allelic exchange or allelic replacement). Standard recombinant DNA techniques such as cloning the gene in a plasmid, digestion of the gene with a restriction enzyme, followed by endonuclease treatment, re-ligation and homologous recombination in the host strain, are all known in the art and described i.a. in Maniatis/Sambrook (Sambrook, J. et al. Molecular cloning: a laboratory manual. ISBN 0-87969-309-6). Site-directed mutations can e.g. be made by means of in vitro site directed mutagenesis using the TRANSFORMER® kit sold by Clontech. PCR-techniques are extensively described in (Dieffenbach & Dreksler; PCR primers, a laboratory manual. ISBN 0-87969-447-5 (1995).

A mutation may be introduced at a predetermined site in genomic DNA via an insertion, a deletion, or a substitution of one nucleotide by another, such as a point mutation with the only proviso that the mutated gene encodes no corresponding Type III secretion system protein, a non-functional corresponding Type III secretion system protein, no corresponding protein encoded by a gene of the cyaA locus, or a non-functional corresponding protein encoded by a gene of the cyaA locus. The mutation should produce a bacteria with no Type III secretion system, a non-functional Type III secretion system, no corresponding protein of the cyaA locus, no cyaA protein, or a non-functional cyaA protein. Preferably, the mutation is a deletion mutation, where disruption of the gene is caused by the excision of nucleic acids. Such a mutation can e.g. be made by deletion of a number of base pairs. Even very small deletions such as stretches of 10 base pairs can already cause the gene to encode no protein or a non-functional protein. Even the deletion of one single base pair may lead to no protein or a non-functional Type III secretion system protein or no protein or a non-functional protein of the cyaA locus, since as a result of such a mutation, the other base pairs are no longer in the correct reading frame or transcription has been inhibited or diminished. More preferably, a longer stretch is removed e.g. 100 base pairs. Even more preferably, the whole gene is deleted. Well-defined and deliberately made mutations involving the deletion of fragments or the whole gene of a gene of the Type III secretion system or a gene of the cyaA locus, e.g. the cyaA gene, or combinations thereof, have the advantage, in comparison to classically induced mutations, that they will not revert to the wild-type situation. Thus, in an even more preferred form, an embodiment of the invention induces live attenuated bacteria in which a mutation in a gene of the Type III secretion system and in a gene of the cyaA locus comprises a deletion or an insertion to disrupt the genes so that no corresponding proteins or non-functional proteins are produced. In one aspect, bacteria having mutations in one or more genes of the Type III secretion system and in one or mores genes of the cyaA locus generate live attenuated double mutant Bordetella bacteria having no Type III secretion system or a non-functional Type III secretion system and no Cya protein or a non-functional CyaA protein.

One skilled in the art will also appreciate that the Bordetella bacteria used in conjunction with the various mutagenesis techniques may be wild type or have a pre-existing mutation in a gene of the Type III secretion system or in a gene of the cyaA locus, e.g. a cyaA gene, and be subjected to further mutagenesis or recombinant DNA techniques to construct double mutant bacteria of the present invention.

Techniques for identifying Bordetella bacteria having one or more mutations in a gene of the Type III secretion system resulting in the lack of expression of the corresponding protein or in the production of a non-functional protein are known by one skilled in the art. Routine techniques for their detection such as Northern and Western blotting, ELISAs and cytotoxicity assays are known in the art and described elsewhere herein. Mutant bacteria with no Type III secretion system or a non-functional Type III system can easily be selected as described elsewhere herein.

Techniques for identifying mutant Bordetella bacteria having one or more mutations in a gene of the cyaA locus resulting in the lack of expression of the corresponding protein or in the production of a non-functional protein are known by one skilled in the art. Routine techniques for their detection such as Northern and Western blotting, ELISAs, enzymatic assays, and hemolysis assays are known in the art and described elsewhere herein. Mutant bacteria with no cyaA protein or a non-functional cyaA protein can easily be selected as described elsewhere herein.

Detection of the double mutants may be by any suitable method, including assays that test for mutations in the Type III secretion system or cyaA locus individually or together or phenotypes resulting from these mutations, e.g. cytotoxicity, hemolysis and pathology assays. The colonies can be selected and grown for vaccine purposes using standard techniques as appreciated by one skilled in the art and as described herein.

### III. Vaccine Compositions

Because of the vaccine's attenuated but immunogenic character in vivo, the vaccine provides effective protection even in immunocompromised subjects and importantly decreased pathology in the lungs compared to infections of subjects infected with the corresponding virulent, wild type Bordetella bacteria. Thus, still another embodiment of the invention relates to live attenuated vaccines for immunizing animals and humans against a disease caused by infection with Bordetella bacteria. Such vaccines comprise an immunizing amount of live attenuated bacteria for use in a vaccine, according to the invention. As used herein, “immunizing amount” refers to the amount of bacteria which will provide immunity to a Bordetella bacterium. The “immunizing amount” will depend upon the species, breed, age, size, health status and whether the animal has previously been given a vaccine against the same organism.

In one aspect, the vaccines of the present invention are avirulent. As used herein, the term “avirulent” is understood to mean that the double mutant Bordetella bacteria have lost their ability to cause disease in a mammal infected with the strain as compared to an originally virulent bacterial strain, e.g. parental strain, from which the double mutant was derived. The vaccines may be avirulent in immunocompetent or immunocompromised mammals.

Administration of the vaccines to uninfected (naïve) subjects is effective to reduce either or both of the death and disease caused by infection of Bordetella. Further, if an uninfected, vaccinated subject is subsequently infected with Bordetella, the vaccine is effective to prevent or decrease the severity of a subsequent infection by Bordetella bacteria in any of the respiratory organs of the respiratory tract, e.g. the upper respiratory tract (nose and nasal passages, throat, sinuses), the lower respiratory tract (lungs), and respiratory airways (larynx, trachea, and bronchi). This can be determined by any number of methods including colony counting, i.e. bacterial number count, wherein a reduction in bacterial numbers in an organ of the respiratory tract, indicates that the vaccine is effective in preventing or lessening the severity of a subsequent infection by wild type Bordetella bacteria. In some cases the reduction in bacterial numbers may be as great as 1000-fold. The vaccine is effective in decreasing pathology of Bordetella in an organ of the respiratory tract, in particular, the lungs, when challenged by wild type Bordetella bacteria. Histopathological evaluation of lung sections may be performed to determine lung inflammation occurring after vaccination, infection, or challenge as compared to a control using standard techniques such as H & E staining to score lung lesions semi-quantitatively. For example, the scores for the lung legions range from absent (O), minimal (1), slight (2), moderate (3), marked (4), or severe (5) per type of lesion and added up to calculate the pathology-score. See, for example, Example 2. The greater the score, the more severe the inflammation of the lungs. Vaccinated mammals challenged with Bordetella infection have lower lung lesion scores as compared with the control mammals. Mammals administered intranasally the vaccines of the double mutant Bordetella bacteria to an immunocompetent mammal have decreased lung pathology as compared to the lung pathology of a control, e.g. a mammal administered intranasally the same amount of the corresponding wild type Bordetella bacteria.

The vaccines of the present invention upon administration are found to decrease colonization or recolonization of Bordetella bacteria in a mammal, for example, in the mammal's organs of the respiratory tract, e.g. the upper respiratory tract, e.g. the nose and nasal passages, throat, sinuses, the lower respiratory tract, e.g. the lungs, or the respiratory airways, e.g. larynx, trachea, and bronchi or combinations thereof. As used herein, “colonization” or “recolonization” refers to the presence of Bordetella bacteria in the respiratory tract of a mammal. The vaccines upon administration to a mammal may colonize the respiratory tract as efficiently as the mutant's wild type parental strain. Typically, the efficiency may be measured by comparing the bacterial number of the double mutant Bordetella bacteria in the respiratory tract as compared to the control, e.g. the bacterial number of the mutant's wild type parental strain, over a certain period of time, for example, the first 1-3 days after administration of the bacteria to a mammal. Advantageously, the vaccines of the present invention may provide cross protection of different Bordetella species. Accordingly, vaccines of the invention may provide cross-protective immunity against other Bordetella in addition to the Bordetella species or strain employed in the vaccine.

Mammals immunized with vaccines of the present invention generate protective antibodies. It is possible to use the mammal's serum as a source of protective antibodies to protect non-immunized mammals against Bordetella infection when this serum is passively transferred in vivo from the immunized mammal to the non-immunized mammal. This is evidenced by reduced bacterial count of the Bordetella bacteria in the lungs and upper and lower respiratory tract upon subsequent challenge with a corresponding virulent wild type Bordetella bacteria. The decrease in bacterial number between the mammal receiving the antibodies and a control may be compared to determine whether the decrease is statistically significant.

In one embodiment, upon immunization of a mammal with the vaccines of the present invention, the mammal generates an antibody response that is as great as the antibody response generated in a mammal administered an amount of corresponding wild type Bordetella bacteria. The mammals immunized may be immunocompromised or immunocompetent. In a preferred embodiment, the vaccines or double mutant Bordetella bacteria are delivered intranasally. Blood may be collected from the immunized and non-immunized naïve mammals for the measurement of serum levels of antibodies to determine titers using ELISA as described in Example 2.

In one embodiment, the mammal is administered a low dose of the vaccine to infect the respiratory tract. The low dose includes a range from about 100 and 1,000,000 colony forming units (CFUs) of the live attenuated double mutant bacteria of the present invention. The dose can be considered a therapeutic or prophylactic dose which is sufficient to treat initial or subsequent Bordetella infections or prevent Bordetella infections.

In one embodiment of the invention, the vaccines upon administration to a mammal generate protective immunity that is as great as the protective immunity generated by the same amount of corresponding wild type Bordetella bacteria upon challenge with Bordetella bacteria. As used herein, the term “protective immunity” means that a vaccine or immunization schedule that is administered to a mammal induces an immune response that prevents, retards the development of, or reduces the severity of a disease that is caused by Bordetella bacteria, or diminishes or altogether eliminates the symptoms of the disease. The phrase “disease caused by infection of Bordetella bacteria” encompasses any clinical symptom or combination of clinical symptoms that are present in an infection with a member of genus of Bordetella bacteria. These symptoms include but are not limited to: fever, sneezing, nasal discharge, submandibular lymphadenopathy, râles, bronchopneumonia, death, paroxysmal coughing, lung lesions, colonization of the upper respiratory tract (nose and nasal passages, throat, sinuses), the lower respiratory tract (lungs), respiratory airways (larynx, trachea, and bronchi), inflammation, failure to gain weight, turbinate atrophy, and lethargy and the like.

As appreciated by one skilled in the art, the vaccine may include an adjuvant or pharmaceutically acceptable carrier or both. Any suitable adjuvant or pharmaceutically acceptable carrier may be used in the present invention. A pharmaceutically acceptable carrier may be as simple as water, but it may, for example, also comprise culture fluid in which the bacteria were cultured. Another suitable carrier is, for example, a solution of physiological salt concentration.

Given the large amount of vaccines given nowadays to both pets and farm animals, it is clear that combined administration of several vaccines would be desirable. It is therefore very attractive to use live attenuated bacteria as a recombinant carrier for heterologous genes, for example, encoding antigens selected from other pathogenic microorganisms or viruses. Administration of such a recombinant carrier has the advantage that immunity is induced against two or more diseases at the same time. Live attenuated bacteria for use in a vaccine according to the present invention provide very suitable carriers for heterologous genes. In principle such heterologous genes can be inserted in the bacterial genome at any non-essential site.

In one embodiment, the present invention includes live attenuated double mutant Bordetella bacteria of the present invention comprising at least one heterologous gene. In one aspect, the heterologous gene encodes an antigen selected from other pathogenic microorganisms or viruses. The attenuated bacteria can therefore act as a delivery vehicle for administering antigens against other bacterial or viral infections.

Antigens which are suitable for use in this way will be apparent to the skilled person and include antigens derived from Leptospira canicola, Leptospira grippotyphosa, Leptospira hardjo, Leptospira ictero-haemorrhagiae, Leptospira pomona, Leptospira interrogans, Leptospira bratislava, canine distemper virus, canine adenovirus type 2, canine parainfluenza virus, canine parvovirus, rabies, herpes viruses, HIV, Erysipelothrix rhusiopathiae, Pasteurella, Pasteurella multocida, Ascaris, Oesophagostomum, pseudorabies virus, porcine parvovirus, pathogenic Escherichia coli, Bacillus anthracis, respiratory syncytial virus, Porcine Reproductive and Respiratory Syndrome Virus (PRRSV), swine influenza virus (SIV), porcine circovirus (PCV), Clostridium, Salmonella, Vibrio, Mycoplasma, Actinobacillus pleuropneumoniae, Haemophilus, rotavirus, transmissible gastroenteritis virus, Streptococcus sobrinus, Streptococcus mutans, and influenza and the like, may be used. Other antigens, and antigens from other pathogens, which may be used in accordance with the present invention are within the skill and knowledge in the art. Bordetella factors, including virulence factors, may also be expressed, preferably in a modified form which prevents their deleterious effects while permitting the elicitation of an immune response specific to those factors.

In one embodiment, the double mutant Bordetella bacteria include inserting a heterologous gene encoding a protein involved in triggering the immune system, such as an interleukin, Tumor Necrosis Factor or an interferon, or another gene involved in immune-regulation.

The use of a gene in the Type III secretion system, a gene of the cyaA locus, e.g. cyaA gene, or both as an insertion site has the advantage that there is no need to find a new insertion site for the heterologous gene or antigen and at the same time the Type III secretion system, cyaA protein, or both is not produced or is rendered non-functional and the newly introduced heterologous gene can be expressed. The construction of such recombinant carriers can be done routinely, using standard molecular biology techniques such as allelic exchange.

The useful dosage to be administered will vary depending on the age, weight and mammal vaccinated, the mode and route of administration and the type of pathogen against which vaccination is sought. The vaccines of the present invention may be used to immunize a broad range of hosts, for example, mammals, including but not limited to humans, mice, rats, guinea pigs, rabbits, opossums, raccoons, cats, dogs, ferrets, foxes, pigs, hedgehogs, sheep, koala, bears, leopards and horses and the like.

The vaccine may comprise any dose of bacteria, sufficient to elicit an immune response. The number of bacteria that are required to be present in the formulations can be determined and optimized by the skilled person. However, in general, the subject mammal may be administered approximately between 10² and 10¹⁰ bacteria. Doses between 10² and 10⁶ bacteria are even more preferred. The bacteria may be administered in a single dosage unit or multiple sequential dosages.

To formulate the vaccine compositions, the double mutant Bordetella bacteria may be present in a composition together with any suitable pharmaceutically acceptable adjuvant, diluent or excipient. Suitable formulations will be apparent to the skilled person. Optionally, one or more compounds having adjuvant activity may be added to the vaccine. Adjuvants are non-specific stimulators of the immune system. They enhance the immune response of the host to the vaccine. Examples of adjuvants known in the art are Freunds Complete and Incomplete adjuvant, vitamin E, non-ionic block polymers, muramyldipeptides, ISCOMs (immune stimulating complexes, cf. for instance European Patent EP 109942), Saponins, mineral oil, vegetable oil, and Carbopol. Adjuvants, specially suitable for mucosal application are e.g. the E. coli heat-labile toxin (LT) or Cholera toxin (CT). Other suitable adjuvants are for example aluminium hydroxide, aluminium phosphate or aluminium oxide, oil-emulsions (e.g. of Bayol F® or Marcol 52®, saponins or vitamin-E solubilisate. Therefore, in a preferred form, the vaccines according to the present invention comprise an adjuvant.

Other examples of pharmaceutically acceptable carriers or diluents useful in the present invention include stabilizers such as SPGA, carbohydrates (e.g. sorbitol, mannitol, starch, sucrose, glucose, dextran), proteins such as albumin or casein, protein containing agents such as bovine serum or skimmed milk and buffers (e.g. phosphate buffer). Especially when such stabilizers are added to the vaccine, the vaccine is very suitable for freeze-drying or spray-drying.

In accordance with the present invention, the live attenuated double mutant Bordetella bacteria of the present invention may be administered by any effective route. The mutants are preferably administered such that the mutant bacteria colonize the respiratory tract. Preferred administration is via inhalation and oral administration of the live attenuated double mutant Bordetella bacteria. The vaccines according to the present invention can be given inter alia intranasally, intradermally, subcutaneously, orally, by aerosol or intramuscularly.

### IV. Immunizing a Mammal Against a Disease Caused by Infection of Bordetella

The invention provides methods for protecting a mammal immunized with a vaccine of the present invention against disease caused by infection of Bordetella bacteria. In accordance with the invention, a method of immunizing a mammal against a disease caused by infection of Bordetella bacteria includes administering a vaccine comprising an immunizing amount of a live attenuated double mutant Bordetella bacteria. In one aspect, the live attenuated double mutant bacteria have mutations in one or more genes of the Type III secretion system and in one or mores genes of the cyaA locus resulting in the production of no Type III secretion system or a non-functional Type III secretion system and no CyaA protein or a non-functional CyaA protein or combinations thereof. Double mutants are described elsewhere herein.

As appreciated by one skilled in the art, administration of the vaccine can be effected by any suitable method, including but not limited to parenteral injection, intranasal administration, intrapharyngeal administration, or topical administration. In accordance with the practice of the invention, the mammal can be a human or an animal that is need of protection against diseases caused by infection of Bordetella bacteria. Exemplary diseases include but are not limited to Feline Bordetellosis, kennel cough, whooping cough (pertussis), rhinitis and/or respiratory disease caused by Bordetella. Mammals in need of protection against diseases caused by infection of Bordetella bacteria include but are not limited to humans, mice, rats, guinea pigs, rabbits, opossums, raccoons, cats, dogs, ferrets, foxes, pigs, hedgehogs, sheep, koala, bears, leopards and horses and the like. With respect to dogs, dogs may be immunized to prevent or protect against kennel cough, a disease caused by infection of Bordetella bronchiseptica bacteria. Suitable means of administering the vaccine will be apparent to one skilled in the art, although intrapharyngeal administration is preferred for the treatment of kennel cough. With respect to swines, swines may be immunized to prevent or protect against atrophic rhinitis and/or turbinate atrophy, diseases caused by infection of Bordetella bronchiseptica bacteria. In the example of atrophic rhinitis and/or turbinate atrophy, the preferred route of administration is intranasal application, although one skilled in the art will appreciate that other administration means are possible. The determination of the dosage of the vaccine to be administered is well within one skilled in the art. Typically, the amount of the bacteria can be in a range of 1 bacterium to 1,000,000 bacteria per administration depending on the route administered, the particular mammal in need of treatment, and the size and health of the subject mammal.

In another embodiment, the invention includes a method of immunizing mammals against a disease caused by infection of Bordetella bacteria and a disease caused by at least one other pathogen comprising administering a vaccine composition that includes an immunizing amount of the vaccine of the present invention comprising the live attenuated double mutant Bordetella bacteria, and an immunizing amount of one or more antigens of another pathogen. Exemplary pathogens that antigens may be derived from include but are not limited to Leptospira canicola, L. grippotyphosa, L. hardjo, L. ictero-haemorrhagiae, L. pomona, L. interrogans, L. bratislava, canine distemper virus, canine adenovirus type 2, canine parainfluenza virus, canine parvovirus, rabies, herpes viruses, HIV, SIV, Erysipelothrix rhusiopathiae, Pasteurella, P. multocida, Ascaris, Oesophago-stomum, pseudorabies virus, porcine parvovirus, pathogenic E. coli, including E. coli having K88, K99,987P, and/or F41 adherence factors, Bacillus anthracis, respiratory syncytial virus, PRRSV, swine influenza virus (SIV), and porcine circovirus (PCV), Clostridium spp., including Cl. perfringens, and Cl. perfringens type C beta toxoid, Salmonella, Vibrio, Mycoplasma, Actinobacillus pleuropneumoniae, Haemophilus, rotavirus, transmissible gastroenteritis virus, Streptococcus sobrinus, S. mutans, influenza, and the like. It is contemplated that the antigen may be expressed by the double mutant or by combining the vaccine of the invention with another vaccine for protection against a disease, disorder or condition caused by another pathogen.

### V. A Method of Treating a Disease Caused by Infection of Bordetella

In another embodiment, a method for treating a disease caused by infection of Bordetella in a mammal includes administering to the mammal an effective amount of a live attenuated double mutant Bordetella bacteria having at least one mutation in one or more genes of the Type III secretion system and in one or mores genes of the cyaA locus. In one aspect, the mutations produce no corresponding Type III secretion system protein or a non-functional corresponding Type III secretion system protein and no corresponding protein encoded by a gene of the cyaA locus or a non-functional corresponding protein encoded by a gene of the cyaA locus or a combination thereof. In one aspect, the mutations result in the production of no Type III secretion system or a non-functional Type III secretion system and no CyaA protein or a non-functional CyaA protein. Double mutants are described elsewhere herein. As used herein, the term “treating” refers to: (i) preventing a disease, disorder or condition from occurring in an animal or human that may be predisposed to the disease, disorder and/or condition but has not yet been diagnosed as having it; (ii) inhibiting the disease, disorder or condition, i.e., arresting its development; and/or (iii) relieving the disease, disorder or condition, i.e., causing regression of the disease, disorder and/or condition. For example, with respect to whooping cough or a respiratory disease caused by Bordetella, treatment may be measured quantitatively or qualitatively to determine the presence/absence of the disease, or its progression or regression using, for example, symptoms associated with the disease or clinical indications associated with the pathology. In one aspect, the effective amount of a mutant Bordetella bacteria is administered with a pharmaceutically acceptable carrier, adjuvant, or both.

As described elsewhere herein, administration of the live attenuated double Bordetella bacteria mutants can be effected by any suitable method, including but not limited to parenteral injection, intranasal administration, intrapharyngeal administration, or topical administration. In accordance with the practice of the invention, the mammal can be a human or an animal that is need of treatment for diseases caused by infection of Bordetella bacteria. Exemplary diseases include but are not limited to Feline Bordetellosis, kennel cough, whooping cough (pertussis), rhinitis and/or respiratory disease caused by Bordetella. Mammals in need of treatment for diseases caused by infection of Bordetella bacteria include but are not limited to humans, mice, rats, guinea pigs, rabbits, opossums, raccoons, cats, dogs, ferrets, foxes, pigs, hedgehogs, sheep, koala, bears, leopards and horses and the like. With respect to dogs, dogs may be treated with live attenuated double Bordetella bacteria mutants of the present invention to treat kennel cough. Means of administering the bacteria are described elsewhere herein, although intrapharyngeal administration is preferred for the treatment of kennel cough. With respect to swines, swines may be administered the live attenuated double Bordetella bacteria mutants to treat atrophic rhinitis and/or turbinate atrophy. In the example of atrophic rhinitis and/or turbinate atrophy, the preferred route of administration is intranasal application, although one skilled in the art will appreciate that other administration means are possible. The determination of the dosage of the vaccine to be administered is well within one skilled in the art. Typically, the amount of the bacteria can be in a range of 1 bacterium to 100 million bacteria per administration depending on the route administered, the particular mammal in need of treatment, and the size and health of the mammal subject.

In another embodiment, the invention includes a method of treating mammals against a disease caused by infection of Bordetella bacteria and a disease caused by at least one other pathogen comprising administering a vaccine composition that includes an effective amount of the vaccine of the present invention comprising the double mutant Bordetella bacteria Type III secretion system/CyaA, and an effective amount of one or more antigens of the pathogen. Exemplary pathogens that antigens may be derived from include but are not limited to Leptospira canicola, L. grippotyphosa, L. hardjo, L. ictero-haemorrhagiae, L. pomona, L. interrogans, L. bratislava, canine distemper virus, canine adenovirus type 2, canine parainfluenza virus, canine parvovirus, rabies, herpes viruses, HIV, SIV, Erysipelothrix rhusiopathiae, Pasteurella, P. multocida, Ascaris, Oesophago-stomum, pseudorabies virus, porcine parvovirus, pathogenic E. coli, including E. coli having K88, K99,987P, and/or F41 adherence factors, Bacillus anthracis, respiratory syncytial virus, PRRSV, swine influenza virus (SIV), and porcine circovirus (PCV), Clostridium spp., including Cl. perfringens, and Cl. perfringens type C beta toxoid, Salmonella, Vibrio, Mycoplasma, Actinobacillus pleuropneumoniae, Haemophilus, rotavirus, transmissible gastroenteritis virus, Streptococcus sobrinus, S. mutans, influenza, and the like. It is contemplated that the antigen may be expressed by the double mutant or by combining the vaccine of the invention with another antigen or vaccine for protection against disease caused by another pathogen.

In another embodiment, the vaccines or live attenuated double Bordetella mutants of the present invention when administered to a mammal may elicit a humoral immune response, cell-mediated response or combinations thereof against Bordetella infections in the mammal. The ability of the vaccines or double Bordetella mutants to elicit a humoral or cell mediated immune response may depend on the amount of the vaccine or bacteria administered, the route of administration, and previous exposure to the Bordetella infections. Appropriate assays and techniques in which to evaluate the type and magnitude of the immune response, include but are not limited to colony counting, ELISA for antibody titer determination, ELISA for cytokine determination, H & E staining for pathology, and challenge of vaccination in in vivo models, e.g. wild type (WT) Balb/c, C3H/HeJ (TLR4^{def}), and TNFα^{-/-}BL/6.129 (TNFα^{-/-}) mice.

The double mutant Bordetella bacteria in which the CyaA protein and Type III secretion system are not produced or are non-functional and vaccines comprising them are available for use as antigens to generate the production of antibodies for use in passive immunotherapy, for example, the adoptive transfer of immune serum from a mammal immunized with a vaccine of the present invention and transferred to a mammal in need thereof, for example, a naïve mammal that is susceptible to Bordetella infection. For example, serum comprising antibodies produced by immunizing a host with double mutant Bordetella bacteria in which the Type III secretion system and CyaA protein are not produced or are non-functional are used for the therapeutic treatment of a disease caused by Bordetella bacterial infection. Thus, the generated serum can be used for either prophylactic or therapeutic applications.

### VI. Preparing a Bordetella Vaccine

The present invention also includes a method of preparing a Bordetella vaccine composition comprising mixing an immunizing amount of a pharmaceutically acceptable carrier and live attenuated double mutant Bordetella bacteria having mutations in one or more genes of the Type III secretion system and in one or mores genes of the cyaA locus, so that the corresponding proteins of the Type III secretion system and cyaA locus are not produced or are non-functional. In one aspect, the live attenuated double mutant Bordetella bacteria have no Type III secretion system or a non-functional Type III secretion system and no CyaA protein or a non-functional CyaA protein.

The vaccine may be prepared from freshly harvested cultures by methods that are standard in the art, for example, the live attenuated double mutant bacteria may be propagated in a culture medium such as Bordet-Gengou agar (Difco) with 10% defibrillated sheep's blood, inoculated into Stainer-Scholte broth at an appropriate optical density, typically of 0.1 or lower, and grown to mid-log phase at 37° C. The growth of the bacteria is monitored by standard techniques and harvested when a sufficient or desired density of the double mutant Bordetella bacteria has been achieved. Other methods, such as those described in Example 2, can be employed.

In another aspect, the present invention includes a method of preparing a vaccine composition for Bordetella and another pathogen comprising mixing an immunizing amount of the live attenuated double mutant Bordetella bacteria with an immunizing amount of one or more antigens of another pathogen, and a pharmaceutically acceptable carrier. The vaccine may include an adjuvant. The vaccine composition may be administered in any suitable manner, including but not limited to intramuscular, subcutaneous, intranasal, intraperitoneal or oral routes, preferably by intranasal routes. In one aspect, the vaccine composition of the present invention advantageously provides immunity from infection after a single administration.

The immunogenicity of the vaccine composition may be tested in any suitable system, using for example, a mammal, such as a human, or an animal, such as a rabbit, pig, rat, dog, cat, mouse, etc. Control animals may be used to test variables, such as vaccine composition dosage. Post-immune serum may be collected from the immunized animal, and the amount of anti-Bordetella antibody present in their serum determined, using for example, an ELISA test. One such procedure is described in Example 1. When the vaccine includes an additional antigen derived from a pathogen, the immunogenicity of that component of the vaccine may be tested as well, for example, by using ELISA to determine the amount of antibody present for that pathogenic antigen.

The following examples are intended to further illustrate the invention without limiting its scope.

### EXAMPLES

### Example 1

### Use of a Genetically Defined Double Mutant strain of B. bronchiseptica Lacking Adenylate Cyclase and Type III Secretion as a Live Vaccine

While most vaccines consisting of killed bacteria induce high serum antibody titers, they do not always confer protection as effective as that induced by infection, particularly against musocal pathogens. Bordetella bronchiseptica is a gram-negative respiratory pathogen that is endemic in many non-human mammalian populations and causes substantial disease in a variety of animals. More than 10 different live attenuated vaccines are available against this pathogen for use in a variety of livestock and companion animals. However, there is little published data on the makeup or efficacy of these vaccines, and each has serious limitations, described above. Here we report the use of AVS, a genetically engineered double mutant of B. bronchiseptica, which lacks adenylate cyclase and type III secretion, as a vaccine candidate. This strain is safe at high doses, meaning it did not cause overt symptoms such as respiratory distress, ruffled fur, failure to gain weight or non-responsiveness, even in highly immunocompromised animals that were rapidly killed by wild type B. bronchiseptica. AVS induces protective immune responses that are able to prevent wild type B. bronchiseptica colonization in the lower respiratory tract and reduce bacterial numbers in the upper respiratory tract, relative to naïve animals, by greater than 1000-fold. This novel B. bronchiseptica vaccine candidate induces strong local immunity while eliminating damage caused by two predominant cytotoxic mechanisms.

### Example 2

### Materials and Methods

### Bacteria.

Bacteria were maintained on Bordet-Gengou agar (Difco) with 10% defibrillated sheep's blood, inoculated into Stainer-Scholte broth at optical densities of 0.1 or lower, and grown to mid-log phase at 37° C. on a roller drum. Wild-type strains of B. bronchiseptica (RB50), B. parapertussis (12822), and B. pertussis (BP536) have been described previously (Cotter, P. A., and J. F. Miller. 1994. BvgAS-mediated signal transduction: analysis of phase-locked regulatory mutants of Bordetella bronchiseptica in a rabbit model. Infect Immun 62:3381-3390; Heininger, U., P. A. Cotter, H. W. Fescemyer, G. Martinez de Tejada, M. H. Yuk, J. F. Miller, and E. T. Harvill. 2002. Comparative phenotypic analysis of the Bordetella parapertussis isolate chosen for genomic sequencing. Infect Immun 70:3777-3784; Relman, D. A., M. Domenighini, E. Tuomanen, R. Rappuoli, and S. Falkow. 1989. Filamentous hemagglutinin of Bordetella pertussis: nucleotide sequence and crucial role in adherence. Proc Natl Acad Sci USA 86:2637-2641). The construction of allelic exchange vectors for deleting the genes encoding adenylate cyclase (Harvill, E. T., P. A. Cotter, M. H. Yuk, and J. F. Miller. 1999. Probing the function of Bordetella bronchiseptica adenylate cyclase toxin by manipulating host immunity. Infect Immun 67:1493-1500) and the ATPase necessary for Type III secretion, have been previously described (Yuk, M. H., E. T. Harvill, P. A. Cotter, and J. F. Miller. 2000. Modulation of host immune responses, induction of apoptosis and inhibition of NF-kappaB activation by the Bordetella type III secretion system. Mol Microbiol 35:991-1004). The mutant AVS described herein containing these two deletions has been previously described and is also known as 8W1 or ΔbscN ΔcyaA. See Harvill, E. T., P. A. Cotter, M. H. Yuk, and J. F. Miller. 1999. Probing the function of Bordetella bronchiseptica adenylate cyclase toxin by manipulating host immunity. Infect Immun 67:1493-1500, for example, FIG. 2. Advantageously, these genetic changes provide a clear identification, so that this strain, for example, can be distinguished from any others by polymerase chain reaction (PCR) and sequencing of the small regions containing the deletions. The strain that has been used has had its entire genome sequenced by the Sanger Center, so every gene is known. Furthermore, the use of mutants with deleted genes prevents the possibility of the bacteria mutating these genes and reverting to a virulent strain.

### Animal Experiments.

Wild type (WT) Balb/c, C3H/HeJ (TLR^{def}), and TNFα^{-/-}BL/6.129 (TNFα^{-/-}) mice were obtained from The Jackson Laboratory. Mice were maintained and treated at the Pennsylvania State University in accordance with IACUC and approved institutional guidelines. To evenly distribute the bacteria throughout the respiratory tract, mice were lightly sedated with isofluorane (Abbott Laboratories) and inoculated by pipetting 50 μl of phosphate-buffered saline (PBS) containing the indicated dose of bacteria onto the tip of the external nares (Harvill, E. T., P. A. Cotter, and J. F. Miller. 1999. Pregenomic comparative analysis between Bordetella bronchiseptica RB50 and Bordetella pertussis tohama I in murine models of respiratory tract infection. Infect Immun 67:6109-6118). For survival curves, groups of 10 mice were infected with the indicated bacteria and were euthanized when they displayed signs of deteriorating lethal bordetellosis, which include ruffled fur, hunched backs and labored breathing, in order to alleviate unnecessary suffering. For time course experiments, groups of four animals were sacrificed at the indicated time point after inoculation. Colonization of respiratory organs was quantified by homogenization of each tissue in PBS, plating onto Bordet-Gengou blood agar containing 20 μg of streptomycin per ml, and colony counting. Low dose intranasal (i.n.) vaccinations were performed by pipetting approximately 5 μl of PBS containing 100 CFU of either RB50, or the double mutant, AVS onto the external nares. Reinfections with the indicated bacteria occurred 49 days post primary infection or vaccination. For passive-transfer experiments, wild-type mice were inoculated with 5×10⁵ CFU of B. bronchiseptica strain RB50 or AVS as described above, and serum was collected on day 49 post-inoculation. Two hundred microliters of pooled convalescent-phase serum was injected intraperitoneally into mice immediately before inoculation.

### Lung Histology.

For lung histology, the trachea and lungs were excised and inflated with 10% formaldehyde. The lungs were then sectioned and stained with hematoxylin and eosin at the Animal Diagnostic Laboratory at the Pennsylvania State University. The sections were scored for pathology by a veterinarian with training and experience in rodent pathology who was blinded to experimental treatment (M.J.K.). A score of 0 indicates no noticeable inflammation or lesions; a score of 1 indicates few or scattered foci affecting less than 10% of the tissue; a score of 2 indicates light, mild aggregates affecting 10-20% of the tissue; a score of 3 indicates moderate, notable, easily visible infiltrates affecting 20-30% of the tissue; a score of 4 indicates heavy, extensive and marked inflammation affecting more than 30% of the tissue.

### ELISAs.

Titers of anti-Bordetella antibody in convalescent-phase serum were determined by enzyme-linked immunosorbent assay. In brief, 96 well plates with adhered heat killed RB50 were probed with the indicated convalescent phase serum. Serum was serially diluted in 1:2 ratios across the plate. Endpoint titer was determined by comparison to similarly treated naïve serum. Specific classes and isotypes of antibodies were determined by using appropriate secondary goat anti-mouse HRP conjugated antibodies (Southern Biotechnology Associates and Pharmingen).

Statistical Analysis. For all experiments, statistical significance was determined using a Student's t-test. P-values of less than or equal to 0.05 are indicated (*).

### Example 3

### Results

### AVS is Avirulent in Susceptible Mouse Strains

The deletion of adenylate cyclase and the ATPase necessary for type III secretion results in the ablation of in vitro cytotoxicity by AVS when compared to the parental wildtype strain, RB50 (Harvill, E. T., P. A. Cotter, M. H. Yuk, and J. F. Miller. 1999. Probing the function of Bordetella bronchiseptica adenylate cyclase toxin by manipulating host immunity. Infect Immun 67:1493-1500; Stockbauer, K. E., A. K. Foreman-Wykert, and J. F. Miller. 2003. Bordetella type III secretion induces caspase 1-independent necrosis. Cell Microbiol 5:123-132), suggesting that this mutant may have attenuated virulence during infection. However, this mutant bacteria has not previously been examined in vivo. To determine if AVS is less virulent during infection we examined the ability of this mutant to cause lethal disease in immunocompromised mice lacking TLR4 or TNFα. Wildtype B. bronchiseptica infection in these immunocompromised mice has been previously described and the role of TLR4 and TNFα in Bordetella infection has been well characterized (Mann, P. B., K. D. Elder, M. J. Kennett, and E. T. Harvill. 2004. Toll-like receptor 4-dependent early elicited tumor necrosis factor alpha expression is critical for innate host defense against Bordetella bronchiseptica. Infect Immun 72:6650-6658; Mann, P. B., M. J. Kennett, and E. T. Harvill. 2004. Toll-like receptor 4 is critical to innate host defense in a murine model of bordetellosis. J Infect Dis 189:833-836). As such, these mice are used to determine the safety of AVS in a known susceptible model without drawing redundant conclusions of the role of TLR4 and TNFα. We intranasally inoculated TLR4^{def} and TNFα^{-/-} mice with 10³, 10⁴, or 10⁵ CFU of RB50 or 10⁵ CFU of AVS in a 50 μL inoculum and observed for signs of severe disease. WT mice given similar doses of RB50 are able to control the disease and clear the bacteria from the lower respiratory tract (Kirimanjeswara, G. S., P. B. Mann, and E. T. Harvill. 2003. Role of antibodies in immunity to Bordetella infections. Infect Immun 71:1719-1724). As previously observed, TLR4^{def} mice and TNFα^{-/-} mice rapidly develop signs of bordetellosis including ruffled fur and hunched backs and succumbed within 7 days following inoculation with as little as 103 CFU of RB50 (FIG. 1). However, TLR4^{def} mice and TNFα^{-/-} mice failed to developed signs of bordetellosis following inoculation with 10⁵ CFU of AVS (FIG. 1) and eliminate the bacteria from the lower respiratory tract by day 49 post infection (data not shown). Therefore, all re-infection experiments are done day 49 post inoculation.

### Example 4

### AVS Induces Less Pathology in Susceptible Mouse Strains

To determine if reduced mortality correlated with decreased pathology, we excised the lungs of mice inoculated with 5×10⁵ CFU of RB50 or AVS on day 3 post inoculation and examined hematoxylin and eosin stained sections. The lungs of WT mice infected with RB50 showed a mean pathology score of 2.6, while those infected with AVS received a score of 1.8 (FIG. 2A). The lungs of TLR4^{def} mice and TNFα^{-/-} mice infected with RB50 received lung pathology scores of 3.3 and 3.6 respectively, while their counterparts infected with AVS were scored at 1.5 and 1.9. As compared to the lungs of AVS infected animals, lungs of TLR4^{def} mice and TNFα^{-/-} mice infected with RB50 contained substantially more lesions that were predominantly neutrophilic in nature (FIG. 2B). These results suggest that the diminished inflammation and lung pathology may contribute to the decreased virulence of AVS in susceptible mouse strains.

### Example 5

### AVS Protects Susceptible Mouse Strains Against Re-Infection

The ability of TLR4^{def} and TNFα−/− mice to eliminate AVS from the lower respiratory tract suggests that these mice are able to generate adaptive immunity to this organism. To test whether this provided protection against subsequent infection with WT B. bronchiseptica, we challenged convalescent and naïve TLR4^{def} and TNFα−/− with 5×10⁵ CFU of RB50 and determined bacterial numbers in the respiratory organs on day 3 post inoculation. The nasal cavities of convalescent and challenged TLR4^{def} mice and TNFα^{-/-} mice contained approximately 10⁴ CFU whereas this same organ in naïve mice contained 10⁸ CFU (FIG. 3). Similarly, the bacterial numbers present in convalescent TLR4^{def} and TNFα^{-/-} mice were near or below the lower limit of detection (˜10 CFU) in the trachea and lungs, while naïve mice harbored approximately 10⁷ CFU in the trachea and 10⁹ CFU in the lungs. Unlike naïve TLR4^{def} and TNFα^{-/-} mice which eventually succumb to infection with RB50, mice previously infected with AVS do not develop lethal disease following challenge with RB50 (data not shown). These results indicate that previous infection with AVS generates adaptive immunity which is capable of limiting bacterial colonization of susceptible mice.

### Example 6

### AVS is Defective in Colonizing the Lower But not the Upper Respiratory Tract

In order to examine the usefulness of AVS as a vaccine strain we sought to better characterize infection of WT mice with this strain. To determine the ability of B. bronchiseptica strain AVS to colonize the respiratory tract of mice as compared to its wild type parental strain RB50, we intranasally inoculated WT mice with 5×10⁵ CFU of either RB50 or AVS in a 50 μL inoculum. Bacterial burdens in the respiratory organs were measured on days 0, 3, 7, 14 and 28 post inoculation. On day 3 post inoculation the bacterial numbers of both RB50 and AVS were approximately 10⁶ CFU in the nasal cavity, 10⁴ CFU in the trachea and 10⁵ CFU in the lungs (FIG. 4). Thereafter, RB50 was recovered in numbers 10 to 1000 times that of AVS in the trachea and lungs while remaining at similar CFUs in the nasal cavity. Although AVS is eliminated from the lower respiratory tract faster than RB50, it persisted in the lungs for several weeks prior to clearance by day 28 post inoculation. By comparison, the aroA mutant vaccine strain was cleared by day 8.

### Example 7

### Antibodies Raised Against AVS are Protective

We have previously demonstrated that protection against B. bronchiseptica infection requires Bordetella specific antibodies, so we investigated the ability of AVS to elicit a serum antibody response (Kirimanjeswara, G. S., P. B. Mann, and E. T. Harvill. 2003. Role of antibodies in immunity to Bordetella infections. Infect Immun 71:1719-1724). We collected serum from WT mice 49 days post inoculation with 5×10⁵ of RB50 or AVS and measured anti-B. bronchiseptica (RB50) titers by ELISA. The overall antibody titers generated in response to infection with AVS were similar to those generated by infection with RB50 (FIG. 5A). Titers of about 10,000 or more may be obtained upon administration of AVS. This is in contrast to previous observations with the aroA mutant vaccine strain, which induces approximately 1% of the antibody response induced by the wild type strain. Additionally, no substantial differences in the titers of various antibody isotypes were observed. These results suggests that AVS induces antibody response that is similar in scope to that induced by RB50.

We have previously demonstrated that adoptive transfer of convalescent phase sera from mice infected with RB50 is sufficient to limit infection of this organism in the lower respiratory tract (Kirimanjeswara, G. S., P. B. Mann, and E. T. Harvill. 2003. Role of antibodies in immunity to Bordetella infections. Infect Immun 71:1719-1724). To measure the protective ability of serum antibodies generated by infection with AVS to protect the lower respiratory tract from colonization with WT B. bronchiseptica, we transferred 200 μL of immune serum obtained from naïve, RB50-, or AVS-infected animals into naïve mice and intranasally challenged the mice with 5×10⁵ CFU of RB50 in a 50 μL inoculum. Mice were euthanized at 3 days post challenge and bacterial burdens were determined as previously described. Passive transfer of immune serum obtained from either RB50 or AVS vaccinated mice was able to reduce the bacterial numbers in naïve mice by approximately 1,000 fold in the lungs (FIG. 5B). These results indicate that the antibodies generated in response to AVS are as protective as those generated in response to the wild type strain. This is in contrast to earlier work with defined B. bronchiseptica strain with a deletion in the aroA gene, which generated substantially smaller antibody response than the wild type strain.

### Example 8

### Low Dose Intranasal Vaccination with AVS Protects Against Subsequent RB50 Infection

To investigate the efficacy of AVS as a possible live vaccine candidate we vaccinated groups of WT mice with a single dose of approximately 100 CFU of RB50 or AVS in a 5 mL volume of PBS. On day 49 post vaccination, vaccinated or naïve mice were challenged intranasally with 5×10⁵ CFU of RB50 in a 50 μL inoculum. Mice were euthanized 3 days post challenge and bacterial burdens were determined as previously described. The bacterial numbers of both vaccinated groups were at or near the detectable threshold in the trachea and lungs (FIG. 6) suggesting vaccination with AVS generates a protective immune response that prevents subsequent infection of the lower respiratory tract. To determine if low dose intranasal vaccination with AVS protects susceptible mouse strains, groups of TLR4^{def} and TNFα^{-/-} mice were vaccinated and then challenged with RB50 as described above. The mice were sacrificed on day 3 post secondary challenge and bacterial burdens were measured (data not shown). Results similar to that of infection induced immunity (FIG. 3) were found. The nasal cavities of vaccinated and challenged TLR4^{def} mice and TNFα^{-/-} mice harbored a 10,000 fold decrease in bacterial load when compared to control mice. The trachea and lungs of vaccinated TLR4^{def} mice and TNFα^{-/-} mice contained less that 20 CFU, while the same organs of control mice contained approximately 10⁶ and 10⁸ CFU respectively. These results indicate that low dose intranasal vaccination with AVS protects susceptible mice from severe infection and suggests that AVS is an efficacious Bordetella vaccine.

### Example 9

### Low Dose Intranasal Vaccination with AVS Induces Protective Immunity against Bordetella pertussis and Bordetella Parapertussis in the Lower Respiratory Tract

To determine if vaccination with AVS generated cross protection against B. pertussis and B. parapertussis, groups of WT mice were vaccinated with a single dose of approximately 100 CFU of AVS as described above. On day 49 post vaccination, the mice were challenged intranasally with 5×10⁵ CFU of B. pertussis or B. parapertussis. The mice were euthanized on day 3 post challenge and bacterial burdens were measured. The nasal cavities, tracheae, and lungs of control mice infected with B. pertussis contained approximately 10⁴, 10³ and 10⁵ CFUs respectively, while the same organs of vaccinated mice contained approximately 10³, 10¹ and 10^{2.5} CFUs (FIG. 7A). Similarly, the nasal cavities, tracheae, and lungs of control mice infected with B. parapertussis had approximately 10⁶, 10⁵ and 10⁶ CFUs respectively, while the same organs of vaccinated mice contained approximately 10⁴, 10¹ and 10¹ CFUs (FIG. 7B). These results suggest that, in addition to protecting against B. bronchiseptica infection, low dose intranasal vaccination with AVS also provides substantial cross immunity to B. pertussis and B. parapertussis.

### Example 10

### Discussion

An effective vaccination program is critical to limiting the spread and impact of highly transmissible respiratory pathogens. Ideal candidates for widely used vaccinations should be safe, effective and easily administered. B. bronchiseptica is endemic in many mammalian populations, and a particularly high incidence of infections is seen in kennels as well as pig farms, where extensive vaccination is used to prevent disease (Goodnow, R. A. 1980. Biology of Bordetella bronchiseptica. Microbiol Rev 44:722-738). Therefore, there is a need for a more efficacious B. bronchiseptica vaccine that provides effective and long-lasting protection with a single administration.

Current vaccines used against B. bronchiseptica are composed of either killed wild type bacterial strains that are administered parenterally, or live attenuated vaccine strains that are intranasally administered. Although the general differences in immunity between infection and vaccination with non-infectious components are still being elucidated, the specific mechanisms of clearance that differ between these two types of immunity during B. bronchiseptica infection have been studied. The results indicate that while vaccination with heat killed B. bronchiseptica administered intraperitoneally induced higher serum antibody responses than intranasal infection, vaccination-induced antibodies are less protective in an adoptive transfer model (Different mechanisms of vaccine-induced and infection-induced immunity to Bordetella bronchiseptica. Microbes Infect. 2007 April; 9(4):442-8. Epub 2007 Jan. 12). Vaccination with heat killed bacteria also provides less protection in the upper respiratory tract than infection-induced immunity (Different mechanisms of vaccine-induced and infection-induced immunity to Bordetella bronchiseptica. Microbes Infect. 2007 April; 9(4):442-8. Epub 2007 Jan. 12.) This suggests that the lack of a strong local memory response leads to the need for repeated vaccinations when parenterally delivered killed vaccines are used. Since protective immunity induced by infection is superior to that induced by vaccination with killed or acellular components, the ideal vaccination should consist of infection with a strain that is lacking defined factors that contribute to pathology but are not required for the generation of protective immunity. It would be further advantageous to have a strain that is defective in an ability that is required for virulence but not immunogenicity, such as the ability to colonize the lower respiratory tract, to induce pathology or to cause systemic infection.

Here we describe the use of a mutant of B. bronchiseptica which lacks adenylate cyclase and Type III secretion, AVS, as a live attenuated vaccine. The AVS strain has many characteristics that make it an ideal candidate for use as a live vaccine. AVS is safe at high doses, even in immunocompromised hosts, as it induces less pathology and mortality but also protects animals against infection and disease caused by the virulent parental strain. For example, in several mouse strains (TNF−/−, TLR4^{d}, RAG1−/− . . . ) the wild type strain kills 100% of the animals at a broad range of doses (from 10,000 to 10,000,000), whereas AVS strain kills 0% of these animals at any of those doses. The protection appears to be mediated by antibodies as serum induced by either RB50 or AVS is sufficient to protect wild type mice against disease and bacterial burden in the lungs following RB50 infection. Apparently, neither adenylate cyclase nor type III secretion are required for the generation of a protective immune response, and the lack of antibodies directed against adenylate cyclase did not decrease the efficacy of immune serum. Since AVS expresses the Type Three Secretion System (TTSS) apparatus; it may contribute to protective antigens without producing TTSS-associated pathology. Together, these data suggest that adenylate cyclase and type III secretion are not required for the generation of protective immune responses against the RB50. While AVS does not survive as long as RB50 in the lower respiratory tract of wild type mice, it does persist in a comparable fashion to RB50 in the upper respiratory tract. This suggests that the ability of AVS to persist in the upper respiratory tract, a feature that attenuated strains with metabolic mutations may lack, could contribute to its ability to protect animals against wild type B. bronchiseptica either by direct competition or by stimulating local immune responses. Even a single low dose, low volume inoculation of AVS administered intranasally was able to protect wild type mice against infection with RB50 as well as wild type strains of B. pertussis and B. parapertussis. This protection against the human associated bordetellae may potentially address the current issues of waning immunity seen in vaccinated human populations (He, Q., M. K. Viljanen, H. Arvilommi, B. Aittanen, and J. Mertsola. 1998. Whooping cough caused by Bordetella pertussis and Bordetella parapertussis in an immunized population. Jama 280:635-637; Mielcarek, N., A. S. Debrie, D. Raze, J. Bertout, C. Rouanet, A. B. Younes, C. Creusy, J. Engle, W. E. Goldman, and C. Locht. 2006. Live attenuated B. pertussis as a single-dose nasal vaccine against whooping cough. PLoS Pathog 2:e65; Watanabe, M., and M. Nagai. 2004. Whooping cough due to Bordetella parapertussis: an unresolved problem. Expert Rev Anti Infect Ther 2:447-454). Current whooping cough vaccination strategies also use bolus injection, as opposed to mucosal immunization, which may contribute to the known ability of B. pertussis and B. parapertussis to avoid humoral immunity (Kirimanjeswara, G. S., P. B. Mann, and E. T. Harvill. 2003. Role of antibodies in immunity to Bordetella infections. Infect Immun 71:1719-1724; Wolfe, D. N., G. S. Kirimanjeswara, and E. T. Harvill. 2005. Clearance of Bordetella parapertussis from the lower respiratory tract requires humoral and cellular immunity. Infect Immun 73:6508-6513) The use of live, attenuated whooping cough vaccines has been recently revitalized for use in humans (Mielcarek, N., A. S. Debrie, D. Raze, J. Bertout, C. Rouanet, A. B. Younes, C. Creusy, J. Engle, W. E. Goldman, and C. Locht. 2006. Live attenuated B. pertussis as a single-dose nasal vaccine against whooping cough. PLoS Pathog 2:e65). A vaccine with proven efficacy against the three classical bordetellae, such as AVS, could potentially replace the current bolus injection vaccination strategies.

The means by which infection with AVS can generate protective immunity may reflect the roles of adenylate cyclase and the Type III secretion system during interactions with host immune cells. Both of these virulence factors play several roles that contribute to the ability of B. bronchiseptica to cause disease. Type III secretion as well as adenylate cyclase cause cell death, which probably prevents phagocytosis of B. bronchiseptica by host cells (Harvill, E. T., P. A. Cotter, M. H. Yuk, and J. F. Miller. 1999. Probing the function of Bordetella bronchiseptica adenylate cyclase toxin by manipulating host immunity. Infect Immun 67:1493-1500; Stockbauer, K. E., A. K. Foreman-Wykert, and J. F. Miller. 2003. Bordetella type III secretion induces caspase 1-independent necrosis. Cell Microbiol 5:123-132). In normal mice, the damage caused by type III secretion-mediated necrosis is controlled, however in immunocompromised mice this can lead to severe pathology and even mortality ((Pilione, M. R., L. M. Agosto, M. J. Kennett, and E. T. Harvill. 2006. CD11b is required for the resolution of inflammation induced by Bordetella bronchiseptica respiratory infection. Cell Microbiol 8:758-768) and Mann & Harvill unpublished data). Therefore, the lack of these two toxins results in decreased lung pathology allowing the safe use of AVS in immunocompromised mice. The type III secretion system also contributes to the long-term persistence of B. bronchiseptica in the lower respiratory of the host by actively inhibiting the generation of protective Th1 responses (Pilione, M. R., and E. T. Harvill. 2006. The Bordetella bronchiseptica type III secretion system inhibits gamma interferon production that is required for efficient antibody-mediated bacterial clearance. Infect Immun 74:1043-1049; Skinner, J. A., M. R. Pilione, H. Shen, E. T. Harvill, and M. H. Yuk. 2005. Bordetella Type III secretion modulates dendritic cell migration resulting in immunosuppression and bacterial persistence. J Immunol 175:4647-4652). This results in the early generation of an anti-inflammatory Th2 response and bacterial persistence in the lungs for the first few weeks of infection. Eventually, a Th1 response is generated, and along with antibodies that bind to B. bronchiseptica, facilitates the clearance of bacteria from the lower respiratory tract. Infection with strains that lack Type III secretion results in the earlier generation of a protective Th1 response and faster bacterial clearance (Pilione, M. R., and E. T. Harvill. 2006. The Bordetella bronchiseptica type III secretion system inhibits gamma interferon production that is required for efficient antibody-mediated bacterial clearance. Infect Immun 74:1043-1049; Skinner, J. A., M. R. Pilione, H. Shen, E. T. Harvill, and M. H. Yuk. 2005. Bordetella type III secretion modulates dendritic cell migration resulting in immunosuppression and bacterial persistence. J Immunol 175:4647-4652). By using a rationally designed vaccine strain that does not express toxins that induce pathology and modulate the immune response, an efficient and effective protective immune response is generated without collateral pathology. The consequence is a highly effective vaccine that is both safe to use and generates a strong protective immune response against subsequent infection.

### Example 11

### Making and Testing of a bscN Deletion in RB50

Creating the knockout construct: 419 bp upstream and the first three codons of the bscN gene were PCR amplified using primers flanked with EcoRI on the 5′ end and BamHI on the 3′ end (F-ATCGAATTCCGGATCAGGCGGAGAAGA (SEQ ID NO: 1) and R-TAAGGATCCCTGACGCATGCCCCTATC (SEQ ID NO: 2) respectively). 420 bp downstream and the last three codons of the bscN gene were PCR amplified using primers flanked with BamHI on the 5′ end and EcoRI on the 3′ end (F-CGCGGATCCGAATCCTAATGGACCTGG (SEQ ID NO: 3) and R-TAGGAATTCTCCAGGCTCTCGCGCAAG (SEQ ID NO: 4) respectively). The PCR conditions were 95 for 5 minutes, 30×(95 30 sec, 56 30 sec, 72 for 1 min), 72 for 5 min. These fragments were PCR purified, RE digested with BamHI, gel purified, and ligated overnight at 4 C. The ligation product was then amplified with the 5′F and 3′R primers as described above. 846 bp product was ligated into the TOPO-TA vector and transformed into chem. comp. DH5a cells. Presence of the insert in TOPO-TA was screened by plasmid extraction from resulting transformants and digestion with EcoRI. The 838 bp insert was digested from TOPO-TA, gel purified and ligated overnight into the Bordetella allelic exchange vector pSS4245 which was RE digested with EcoRI and gel purified. The ligation product was transformed as described above. Presence of the insert in pSS4245 was screened by plasmid extraction from resulting transformants and digestion with EcoRI. The positive clone was renamed pSS4245ΔbscN. The resulting positive clones were sequenced after insertion into TOPO-TA and pSS4245.

Mating, Resolving, Screening of KO: DH5a harboring pSS4545ΔbscN or pSS1827 (a plasmid competent for mating) and Bordetella bronchiseptica strain RB50 that was modulated into Bvg-conditions using 50 mM MgSO4 was mated for 4 hours on a BG+10 mM+MgS04 plate at 37 C. RB50 containing pSS4245ΔbscN was selected positively selected for using BG+strep+kan+50 mM MgS04 plates and incubated for 5 days at 37 C. The plasmid was selected against by restreaking colonies on BG+step plates and incubating 2 days resulting in colonies containing either the wild-type or knockout gene and absence of pSS4245. Colonies were screened for the presence either the wild-type or knockout gene by using the 5° F. and 3′R primers as described above with the PCR conditions of 95 for 5 minutes, 30× (95 30 sec, 56 30 sec, 72 2 min), 72 5 min. The wild-type gene was indicated by a 2190 bp band. The knockout was indicated by a 846 bp band. The absence of pSS4245 was confirmed by growth on BG+strep plates and lack of growth on BG+kan plates.

### Example 12

### Making and Testing of Double Mutant Bordetella Bacteria with Additional Mutations in the Type Six Secretion System

We have identified within the B. bronchiseptica genome genes with homology to the type six secretion system (T6SS) of E. coli. Based on published analyses of the genes required for the function of the T6SS in E. coli, we selected a single gene that is required for T6SS-mediated secretion in E. coli and deleted the B. bronchiseptica form of this gene, named BB0810, from the genome of wild type strain RB50. (Comparative analysis of the genome sequences of Bordetella pertussis, Bordetella parapertussis and Bordetella bronchiseptica. Nat. Genet. 2003 September; 35(1):32-40. Epub 2003 Aug. 10.) This strain with this deletion, named BB0810, grows normally in vitro on standard growth media, and is indistinguishable from the wild type strain in its colonization, growth and persistence in the mouse nose, trachea and lungs (data not shown). However, this strain is completely avirulent in TNFα^{-/-} mice; it induces no obvious signs of disease for >100 days, whereas the wild type strain rapidly kills these mice in about 3 days. This phenotype is consistent with in vitro cytotoxicity assays, which show that the BB0810 mutant is decreased in cytotoxicity for both Raw cells and J774 cells, relative to the wild type parental strain RB50. Together these data indicate that the gene BB0810, and presumably the T6SS that it appears to be a vital component of, is not required for efficient infection, but is required for cytotoxicity in vitro and for full virulence in immunocompromised (TNFα^{-/-}) mice. These are characteristics that should not decrease the immunogenicity of the bacterium by decreasing its ability to colonize and grow in its host. They should, however, increase the safety of the strain by eliminating a mechanism that is required only for the most severe pathology. Thus the addition of this mutation to a candidate live vaccine strain should further improve its safety without affecting its immunogenicity and efficacy against virulent forms of B. bronchiseptica.

### Example 13

### Making and Testing of Double Mutant Bordetella Bacteria with Additional Mutations in the Sigma Factor SigE Gene

Sigma E is an alternative sigma factor that facilitates a variety of responses to different stress conditions in a wide variety of bacteria. We have deleted the gene encoding SigmaE, sigE, from the genome of RB50 to determine its role in the biology of B. bronchiseptica. In initial experiments we have observed that the sigE mutant grows normally under standard growth conditions in vitro, but is defective in growth under stress conditions including heat shock or exposure to ethanol. The sigE mutant was indistinguishable from the parental strain, RB50, in its ability to colonize, grow and persist in wild type mice (data not shown). However, the sigE mutant was decreased in cytotoxicity for J774 cells in vitro (FIG. 8B). In addition, this strain was completely avirulent in Ragl^{-/-} mice; while the wild type strain kills 100% of these mice in about 25 days, the sigE mutant did not kill any mice, or cause any signs of disease, for more than 100 days. These data indicate that sigE is not required for the normal infectious process, but is required for the most virulent form of B. bronchiseptica disease. Thus deleting sigE should result in a live vaccine strain that is safer without reduction in immunogenicity that requires efficient infection. The observed defects in growth under heat shock and ethanol exposure further improve the safety in vitro, and may also inhibit the potential for environmental survival and/or transmission between hosts.

Bacterial strains and growth. B. bronchiseptica strain RB50 has been previously described and the isogenic mutant lacking SigE RB50ΔsigE (SigE deficient) was made in this study. Bacteria were maintained on Bordet-Gengou agar (Difco) containing 10% defibrinated sheep blood (Hema Resources) and 20 μg/ml streptomycin. Liquid culture bacteria were grown overnight on a roller drum at 37° C. to mid-log phase in Stainer-Scholte broth (Stainer J Gen Microbio 1970). For the growth curves under stress conditions, stationary phase overnight cultures were subcultured into fresh Stainer-Scholte broth with 1.5% ethanol and bacterial growth were monitored by measuring OD and plating followed by colony counts.

Construction of RB50ΔsigE strain. A left-flanking PCR product with 637 bp proximal to the sigE gene and a non-overlapping 534 bp right-flanking product from B. bronchiseptica RB50 (Parkhill 2003) genomic DNA were amplified. The two flanking fragments were ligated, PCR amplified, cloned into TopoTA vector (invitrogen) and verified by sequencing. The knock-out construct was then cloned into EcoRI site of pSS3962 allelic exchange vector, resulting in the deletion of the central region of sigE. Only 66 bp of the 5′ end and 6 bp of the 3′ end of the sigE gene remained in place, while a 523 bp central region was deleted. Followed by triparental mating and selection steps, sigE was deleted on RB50 chromosome. The knockout strain was verified by colony PCR and southern blot.

Cytotoxicity assays may be performed as described in Harvill, E. T., P. A. Cotter, et al. (1999). “Probing the function of Bordetella bronchiseptica adenylate cyclase toxin by manipulating host immunity.” Infect Immun 67(3): 1493-500, herein incorporated by reference in its entirety.

Animal experiments. C57BL/6, HEJ, TNFα^{-/-} and RAG1^{-/-} mice were obtained from Jackson laboratories (Bar Harbor, Me., USA). All mice were bred in our Bordetella-free, specific pathogen-free breeding rooms at The Pennsylvania State University. For inoculation, mice were sedated with 5% isoflurane (Abbott laboratory) in oxygen and inoculated by pipetting 50 μl of PBS containing 5×10⁵ CFU of bacteria onto the external nares (Kirimanjeswara, G. S. JCI 2005). This method reliably distributes the bacteria throughout the respiratory tract (Harvill I&I 2000). Survival curves were generated by infection of HEJ, TNFα^{-/-} and RAG1^{-/-} mice with either RB50 or RB50ΔsigE. Mice suffering from lethal bordetellosis as determined by severe hunched posture, ruffled fur, extremely labored breathing and apathy were euthanized to prevent unnecessary suffering (Mann I&I 2007). All protocols were reviewed by the university IACUC and all animals were handled in accordance with institutional guidelines.

Bacterial quantification. Mice were sacrificed via CO₂ inhalation and lungs, tracheae, nasal cavities, spleens, livers and/or kidneys, as indicated. Tissues were homogenized in PBS, plated at specific dilutions onto Bordet-Gengou agar containing 20 μg/mL streptomycin, incubated at 37° C. for 3 to 4 days followed by a colony count (Kirimanjeswara, G. S. JCI 2005). The lower limit of detection was 10 CFU and is indicated as the lower limit of the Y-axes.

Statistical Analysis. The mean +/−standard error of the geometric mean was determined for all appropriate data and was expressed as error bars. Two-tailed, unpaired Student's T-tests were used to determine statistical significance between groups. All experiments were performed at least twice with similar results.

### Example 14

### cyaA Deletion

As described in Probing the function of Bordetella bronchiseptica adenylate cyclase toxin by manipulating host immunity. Infect Immun. (1999) 67:1493-1500, pDelta cyaA was constructed by cloning a 5.5-kb BamHI-BsmI fragment encompassing cyaA from B. pertussis into our allelic exchange vector (Cotter, P. A., and J. F. Miller. 1994. BvgAS-mediated signal transduction: analysis of phase-locked regulatory mutants of Bordetella bronchiseptica in a rabbit model. Infect Immun 62:3381-3390), digesting it with ApaLI, and religating it to delete the two consecutive ApaLI fragments which encode the central 1,580 codons of cyaA. The 766-bp BamHI-BsmI fragment remaining in pDelta cyaA contains 67 bp of cyaA promoter sequence, the first 61 codons, the last 65 codons, and 206 bp 3′ to the cyaA stop codon. Delivery of this allele to the chromosome of RB50 (wt) by two consecutive homologous recombination events resulted in construction of strain RB58 (Delta cyaA). Southern hybridization analysis confirmed that RB58 was constructed as intended (data not shown). In vitro assays, performed as previously described (Hewlett E, Wolff J. Soluble adenylate cyclase from the culture medium of Bordetella pertussis: purification and characterization. J. Bacteriol. 1976 August; 127(2):890-898), confirmed that supernatants from RB50, but not RB58, contained adenylate cyclase activity. WD3 (Delta bscN) and 8W1 (Delta bscNDelta cyaA) were constructed as in-frame deletions in bscN in RB50 and RB58, respectively, as previously described (Yuk, M. H., Harvill, E. T., Miller J. F. The BvgAS Virulence Control System Regulates Type III Secretion in Bordetella Bronchiseptica. Mol. Microbiol. 1998 June; 28(5):945-59). RB54 was similarly constructed as an in-frame deletion in bvgS as previously described (Cotter, P. A., and J. F. Miller. 1994. BvgAS-mediated signal transduction: analysis of phase-locked regulatory mutants of Bordetella bronchiseptica in a rabbit model. Infect Immun 62:3381-3390).

### Example 15

### Bacterial Conjugations, Allelic Exchanges, Plasmid Rescues and Construction of In-Frame Deletion

Allelic exchanges were performed using suicide vectors pEG7 or pEGBR (Akerley et al., 1995; Martinez de Tejada et al., 1996; Cotter and Miller, 1997). DNA fragments used for homologous recombinations were subcloned into the vectors and then transformed into E. coli SM10 for mating to B. bronchiseptica. Matings, selection for gentamicin- or kanamycin-resistant co-integrants and counterselection against sucrose sensitivity for second recombination events were performed as described previously (Akerley et al., 1995; Martinez de Tejada et al., 1996; Cotter and Miller, 1997). DNA flanking the original fragment of bscN (from arbitrary-primed PCR) was isolated as follows: the 420 bp PCR fragment was subcloned into pEG7, and the resulting suicide plasmid was introduced into RB50. Genomic DNA from gentamicin-resistant colonies (containing integrated plasmid by homologous recombination into the bscN gene) was digested with NsiI (one of several restriction enzymes used that does not cut within pEG7), self-ligated, transformed into E. coli XL1-Blue and selected by ampicillin resistance. The rescued plasmid containing an extra 4 kb of DNA was restriction mapped, and fragments were subcloned into pBluescript for DNA sequencing on both strands. The assembled sequence was analysed for ORFs and searched for homologous sequences in the database using BLAST (NCBI), and sequence alignments were performed with ALIGN in the FASTA program (University of Virginia). For the construction of the in-frame deletion in bscN, two PCR fragments using primers W1+W2, which amplify a 350 bp fragment (from codon number 54 to codon number 170 of the bscN ORF), and primers W3+W4, which amplify a 420 bp fragment (from codons 262 to 400), were ligated by overlapping PCR, using overlapping regions between W2 and W3, in the presence of primers W1 and W4. Pfu polymerase (Stratagene) was used for these PCRs. The resultant 770 bp fragment was sequenced to ensure the maintenance of the reading frame and then subcloned into pEGBR. The resulting suicide vector was introduced into RB50, and two recombination events were selected for (first by kanamycin resistance and then by sucrose resistance). The resulting colonies were screened by PCR with primers W1 and W4, which give a 770 bp product from the genome of the deletion strain WD3 but a 1050 bp product from the wild type. For the construction of the transcriptional lacZ fusion with bscN, the 420 bp PCR product from W3+W4 was subcloned into the suicide vector pEGZ (Martinez de Tejada et al., 1996), integrated into RB50 genome by homologous recombination and selected by gentamicin resistance. See Yuk, M. H., Harvill, E. T., Miller J. F. The BvgAS Virulence Control System Regulates Type III Secretion in Bordetella Bronchiseptica. Mol. Microbiol. 1998 June; 28(5):945-59), herein incorporated by reference in its entirety.

### REFERENCES

1. Bey, R. F., F. J. Shade, R. A. Goodnow, and R. C. Johnson. 1981. Intranasal vaccination of dogs with liver avirulent Bordetella bronchiseptica: correlation of serum agglutination titer and the formation of secretory IgA with protection against experimentally induced infectious tracheobronchitis. Am J Vet Res 42:1130-1132.
2. Cotter, P. A., and J. F. Miller. 1994. BvgAS-mediated signal transduction: analysis of phase-locked regulatory mutants of Bordetella bronchiseptica in a rabbit model. Infect Immun 62:3381-3390.
3. Edinboro, C. H., M. P. Ward, and L. T. Glickman. 2004. A placebo-controlled trial of two intranasal vaccines to prevent tracheobronchitis (kennel cough) in dogs entering a humane shelter. Prev Vet Med 62:89-99.
4. Goodnow, R. A. 1980. Biology of Bordetella bronchiseptica. Microbiol Rev 44:722-738.
5. Harvill, E. T., P. A. Cotter, and J. F. Miller. 1999. Pregenomic comparative analysis between bordetella bronchiseptica RB50 and Bordetella pertussis tohama I in murine models of respiratory tract infection. Infect Immun 67:6109-6118.
6. Harvill, E. T., P. A. Cotter, M. H. Yuk, and J. F. Miller. 1999. Probing the function of Bordetella bronchiseptica adenylate cyclase toxin by manipulating host immunity. Infect Immun 67:1493-1500.
7. He, Q., M. K. Viljanen, H. Arvilommi, B. Aittanen, and J. Mertsola. 1998. Whooping cough caused by Bordetella pertussis and Bordetella parapertussis in an immunized population. Jama 280:635-637.
8. Heininger, U., P. A. Cotter, H. W. Fescemyer, G. Martinez de Tejada, M. H. Yuk, J. F. Miller, and E. T. Harvill. 2002. Comparative phenotypic analysis of the Bordetella parapertussis isolate chosen for genomic sequencing. Infect Immun 70:3777-3784.
9. Jacobs, A. A., R. P. Theelen, R. Jaspers, L. J. Horspool, D. Sutton, J. G. Bergman, and G. Paul. 2005. Protection of dogs for 13 months against Bordetella bronchiseptica and canine parainfluenza virus with a modified live vaccine. Vet Rec 157:19-23.
10. Kirimanjeswara, G. S., P. B. Mann, and E. T. Harvill. 2003. Role of antibodies in immunity to Bordetella infections. Infect immun 71:1719-1724.
11. Mann, P. B., K. D. Elder, M. J. Kennett, and E. T. Harvill. 2004. Toll-like receptor 4-dependent early elicited tumor necrosis factor alpha expression is critical for innate host defense against Bordetella bronchiseptica. Infect Immun 72:6650-6658.
12. Mann, P. B., M. J. Kennett, and E. T. Harvill. 2004. Toll-like receptor 4 is critical to innate host defense in a murine model of bordetellosis. J Infect Dis 189:833-836.
13. McArthur, J. D., N. P. West, J. N. Cole, H. Jungnitz, C. A. Guzman, J. Chin, P. R. Lehrbach, S. P. Djordjevic, and M. J. Walker. 2003. An aromatic amino acid auxotrophic mutant of Bordetella bronchiseptica is attenuated and immunogenic in a mouse model of infection. FEMS Microbiol Lett 221:7-16.
14. Mielcarek, N., A. S. Debrie, D. Raze, J. Bertout, C. Rouanet, A. B. Younes, C. Creusy, J. Engle, W. E. Goldman, and C. Locht. 2006. Live attenuated B. pertussis as a single-dose nasal vaccine against whooping cough. PLoS Pathog 2:e65.
15. Moylett, E. H., and I. C. Hanson. 2003. 29. Immunization. J Allergy Clin Immunol 111:S754-765.
16. Neutra, M. R., and P. A. Kozlowski. 2006. Mucosal vaccines: the promise and the challenge. Nat Rev Immunol 6:148-158.
17. Pilione, M. R., L. M. Agosto, M. J. Kennett, and E. T. Harvill. 2006. CD11b is required for the resolution of inflammation induced by Bordetella bronchiseptica respiratory infection. Cell Microbiol 8:758-768.
18. Pilione, M. R., and E. T. Harvill. 2006. The Bordetella bronchiseptica type III secretion system inhibits gamma interferon production that is required for efficient antibody-mediated bacterial clearance. Infect Immun 74:1043-1049.
19. Raupach, B., and S. H. Kaufmann. 2001. Bacterial virulence, proinflammatory cytokines and host immunity: how to choose the appropriate Salmonella vaccine strain? Microbes Infect 3:1261-1269.
20. Relman, D. A., M. Domenighini, E. Tuomanen, R. Rappuoli, and S. Falkow. 1989. Filamentous hemagglutinin of Bordetella pertussis: nucleotide sequence and crucial role in adherence. Proc Natl Acad Sci USA 86:2637-2641.
21. Roberts, M., D. Maskell, P. Novotny, and G. Dougan. 1990. Construction and characterization in vivo of Bordetella pertussis aroA mutants. Infect Immun 58:732-739.
22. Skinner, J. A., M. R. Pilione, H. Shen, E. T. Harvill, and M. H. Yuk. 2005. Bordetella type III secretion modulates dendritic cell migration resulting in immunosuppression and bacterial persistence. J Immunol 175:4647-4652.
23. Stevenson, A., and M. Roberts. 2004. Intranasal immunisation against tetanus with an attenuated Bordetella bronchiseptica vector expressing FrgC: improved immunogenicity using a Bvg-regulated promoter to express FrgC. Vaccine 22:4300-4305.
24. Stevenson, A., and M. Roberts. 2002. Use of a rationally attenuated Bordetella bronchiseptica as a live mucosal vaccine and vector for heterologous antigens. Vaccine 20:2325-2335.
25. Stevenson, A., and M. Roberts. 2003. Use of Bordetella bronchiseptica and Bordetella pertussis as live vaccines and vectors for heterologous antigens. FEMS Immunol Med Microbiol 37:121-128.
26. Stockbauer, K. E., A. K. Foreman-Wykert, and J. F. Miller. 2003. Bordetella type III secretion induces caspase 1-independent necrosis. Cell Microbiol 5:123-132.
27. Watanabe, M., and M. Nagai. 2004. Whooping cough due to Bordetella parapertussis: an unresolved problem. Expert Rev Anti Infect Ther 2:447-454.
28. Wolfe, D. N., G. S. Kirimanjeswara, and E. T. Harvill. 2005. Clearance of Bordetella parapertussis from the lower respiratory tract requires humoral and cellular immunity. Infect Immun 73:6508-6513.
29. Yuk, M. H., E. T. Harvill, P. A. Cotter, and J. F. Miller. 2000. Modulation of host immune responses, induction of apoptosis and inhibition of NF-kappaB activation by the Bordetella type III secretion system. Mol Microbiol 35:991-1004.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A method of immunizing a mammal against a disease caused by infection of Bordetella bacteria comprising:
administering to a susceptible mammal an immunizing amount of a vaccine comprising double mutant Bordetella bacteria, wherein at least one gene of the Type III secretion system and at least one gene of the adenylate cyclase toxin (CyaA) locus each comprise at least one mutation so that the Bordetella bacteria produces no Type III secretion system or a non-functional Type III secretion system and no CyaA protein or a non-functional CyaA protein, and a pharmaceutically acceptable carrier.

2. The method of claim 1 wherein the Bordetella bacteria is selected from the group consisting of Bordetella ansorpii, Bordetella avium, Bordetella pertussis, Bordetella parapertussis, Bordetella bronchiseptica, Bordetella avium, Bordetella holmesii, Bordetella petrii, Bordetella trematum, and Bordetella hinzii.

3. The method of claim 2, wherein the Bordetella bacteria is selected from the group consisting of AVS (8W1).

4. The method of claim 1, wherein the immunizing of the mammal further comprises administering the vaccine intradermally, intranasally, intramuscularly, orally, or subcutaneously or administering serum obtained from a mammal administered the vaccine to a susceptible naïve mammal by in vivo passive transfer.

5. The method of claim 1 wherein the administering of said vaccine to the mammal provides cross-protective immunity against Bordetella bacteria.

6. The method of claim 1 further comprising administering at least about 100 CFU of the double mutant Bordetella bacteria to the mammal.

7. The method of claim 1 wherein the administering of said vaccine after a single administration elicits protective immunity from Bordetella bacteria.

8. The method of claim 1 wherein the mammal is selected from the group consisting of humans, mice, rats, guinea pigs, rabbits, opossums, raccoons, cats, dogs, ferrets, foxes, pigs, hedgehogs, sheep, koala, bears, leopards and horses.

9. The method of claim 1 wherein the double mutant Bordetella bacteria further comprises a heterologous gene that encodes an antigen derived from a pathogen selected from the group consisting of Leptospira canicola, Leptospira grippotyphosa, Leptospira hardjo, Leptospira ictero-haemorrhagiae, Leptospira pomona, Leptospira interrogans, Leptospira bratislava, canine distemper virus, canine adenovirus type 2, canine parainfluenza virus, canine parvovirus, rabies, herpes viruses, HIV, Erysipelothrix rhusiopathiae, Pasteurella, Pasteurella multocida, Ascaris, Oesophagostomum, pseudorabies virus, porcine parvovirus, pathogenic Escherichia coli, Bacillus anthracis, respiratory syncytial virus, Porcine Reproductive and Respiratory Syndrome Virus (PRRSV), swine influenza virus (SIV), porcine circovirus (PCV), Clostridium, Salmonella, Vibrio, Mycoplasma, Actinobacillus pleuropneumoniae, Haemophilus, rotavirus, transmissible gastroenteritis virus, Streptococcus sobrinus, Streptococcus mutans, and influenza.

10. The method of claim 9 wherein administration of the double mutant Bordetella bacteria immunizes the mammal against a disease caused by the pathogen from which the antigen was derived.

11. A method for treating a disease caused by infection of Bordetella bacteria in a mammal comprising:
administering to a susceptible mammal an effective amount of double mutant Bordetella bacteria, wherein at least one gene of the Type III secretion system and at least one gene of the adenylate cyclase toxin (CyaA) locus each comprise at least one mutation so that the Bordetella bacteria produces no Type III secretion system or a non-functional Type III secretion system and no CyaA protein or a non-functional CyaA protein.

12. The method of claim 11, wherein the Bordetella bacteria is selected from the group consisting of Bordetella ansorpii, Bordetella avium, Bordetella pertussis, Bordetella parapertussis, Bordetella bronchiseptica, Bordetella avium, Bordetella holmesii, Bordetella petrii, Bordetella trematum, and Bordetella hinzii.

13. The method of claim 12, wherein the Bordetella bacteria is selected from the group consisting of AVS (8W1).

14. The method of claim 11, wherein the treating of the disease caused by infection of Bordetella bacteria in the mammal further comprises administering the double mutant Bordetella bacteria intradermally, intranasally, intramuscularly, orally, or subcutaneously or administering serum obtained from a mammal administered the double mutant Bordetella bacteria to a susceptible naïve mammal by in vivo passive transfer.

15. The method of claim 11, further comprising administering at least about 100 CFU of the double mutant Bordetella bacteria to the mammal.

16. The method of claim 11, wherein the administering of the double mutant Bordetella to a mammal stimulates at least one immune response selected from the group consisting of an antibody response and a cell-mediated immune response.

17. The method of claim 11, wherein the mammal is selected from the group consisting of humans, mice, rats, guinea pigs, rabbits, opossums, raccoons, cats, dogs, ferrets, foxes, pigs, hedgehogs, sheep, koala, bears, leopards and horses.

18. The method of claim 11, wherein the double mutant Bordetella bacteria further comprises a heterologous gene that encodes an antigen derived from a pathogen selected from the group consisting of Leptospira canicola, Leptospira grippotyphosa, Leptospira hardjo, Leptospira ictero-haemorrhagiae, Leptospira pomona, Leptospira interrogans, Leptospira bratislava, canine distemper virus, canine adenovirus type 2, canine parainfluenza virus, canine parvovirus, rabies, herpes viruses, HIV, Erysipelothrix rhusiopathiae, Pasteurella, Pasteurella multocida, Ascaris, Oesophagostomum, pseudorabies virus, porcine parvovirus, pathogenic Escherichia coli, Bacillus anthracis, respiratory syncytial virus, Porcine Reproductive and Respiratory Syndrome Virus (PRRSV), swine influenza virus (SIV), porcine circovirus (PCV), Clostridium, Salmonella, Vibrio, Mycoplasma, Actinobacillus pleuropneumoniae, Haemophilus, rotavirus, transmissible gastroenteritis virus, Streptococcus sobrinus, Streptococcus mutans, and influenza.

19. The method of claim 18 wherein administration of the double mutant Bordetella bacteria treats a disease caused by the pathogen from which the antigen was derived.

20. A method of preparing a Bordetella vaccine composition comprising:
mixing an immunizing amount of a vaccine comprising double mutant Bordetella bacteria, wherein at least one gene of the Type III secretion system and at least one gene of adenylate cyclase toxin (CyaA) locus each comprise at least one mutation so that the Bordetella bacteria produces no Type III secretion system or a non-functional Type III secretion system and no CyaA protein or a non-functional CyaA protein, and a pharmaceutically acceptable carrier.

21. A live attenuated vaccine composition for immunizing a mammal against diseases caused by infection of Bordetella bacteria comprising:
an immunizing amount of a double mutant Bordetella bacteria, wherein at least one gene of the Type III secretion system and at least one gene of the adenylate cyclase toxin (CyaA) locus each comprise at least one mutation so that the Bordetella bacteria produces no Type III secretion system or a non-functional Type III secretion system and no CyaA protein or a non-functional CyaA protein, and a pharmaceutically acceptable carrier.

22. The vaccine composition of claim 21 wherein the Bordetella bacteria is selected from the group consisting of Bordetella ansorpii, Bordetella avium, Bordetella pertussis, Bordetella parapertussis, Bordetella bronchiseptica, Bordetella avium, Bordetella holmesii, Bordetella petrii, Bordetella trematum, and Bordetella hinzii.

23. The vaccine composition of claim 21 wherein the Bordetella bacteria is selected from the group consisting of AVS (8W1).

24. The vaccine composition of claim 21 wherein the gene of the Type III secretion system is selected from the group consisting of bscV, bcr3, bopN, bsp22, bcrHl, bopD, bopB, bcrH2, bcr4, bscl, bscj, bscK, bscL, bscN, and bscO genes.

25. The vaccine composition of claim 21 wherein the gene of the cyaA locus is selected from the group consisting of cyaA, cyaB, cyaC, and cyaD genes.

26. The vaccine composition of claim 21 wherein the mutation in the gene of the Type III secretion system is effected by recombinant DNA technology.

27. The vaccine composition of claim 21 wherein the mutation in the gene of the cyaA locus is effected by recombinant DNA technology.

28. The vaccine composition of claim 21, wherein said double mutant Bordetella bacteria are avirulent.

29. The vaccine composition of claim 21, wherein administration of the vaccine composition to a mammal is effective to prevent a substantial subsequent Bordetella bacterial infection in the lower respiratory tract of the mammal.

30. The vaccine composition of claim 21, wherein administration of the vaccine composition to a mammal provides cross-protective immunity against Bordetella bacteria other than the Bordetella bacteria in the vaccine composition.

31. The vaccine composition of claim 21, wherein administration of about 100 CFU of said double mutant Bordetella bacteria immunizes a susceptible mammal against Bordetella challenge.

32. The vaccine composition of claim 21, wherein serum obtained from a mammal administered the vaccine composition upon administration to a susceptible naïve mammal by in vivo passive transfer provides protection against a disease caused by infection of Bordetella bacteria.

33. The vaccine composition of claim 21, wherein the vaccine composition upon administration to a mammal results in substantially less inflammation in the lungs as compared to inflammation caused by infection by wild type Bordetella bacteria.

34. The vaccine composition of claim 21, wherein said double mutant Bordetella bacteria colonizes the respiratory tract as efficiently as the mutant's wild type parental strain.

35. The vaccine composition of claim 21, wherein the vaccine composition upon intranasal administration to a mammal generates an antibody response that is greater than or equal to an antibody response generated upon intranasal administration to a mammal the same amount of corresponding wild type Bordetella bacteria.

36. The vaccine composition of claim 21, wherein said vaccine composition after a single administration elicits protective immunity from Bordetella bacteria.

37. The vaccine composition of claim 21, which upon administration to a mammal stimulates at least one immune response selected from the group consisting of an antibody response and a cell-mediated immune response.

38. The vaccine composition of claim 21 further comprising an adjuvant.

39. The vaccine composition of claim 21, wherein the double mutant Bordetella bacteria further comprises a heterologous gene.

40. The vaccine composition of claim 39, wherein the double mutant Bordetella bacteria further comprises a heterologous gene that encodes an antigen derived from a pathogen selected from the group consisting of Leptospira canicola, Leptospira grippotyphosa, Leptospira hardjo, Leptospira ictero-haemorrhagiae, Leptospira pomona, Leptospira interrogans, Leptospir bratislava, canine distemper virus, canine adenovirus type 2, canine parainfluenza virus, canine parvovirus, rabies, herpes viruses, HIV, Erysipelothrix rhusiopathiae, Pasteurella, Pasteurella multocida, Ascaris, Oesophagostomum, pseudorabies virus, porcine parvovirus, pathogenic Escherichia coli, Bacillus anthracis, respiratory syncytial virus, Porcine Reproductive and Respiratory Syndrome Virus (PRRSV), swine influenza virus (SIV), porcine circovirus (PCV), Clostridium, Salmonella, Vibrio, Mycoplasma, Actinobacillus pleuropneumoniae, Haemophilus, rotavirus, transmissible gastroenteritis virus, Streptococcus sobrinus, Streptococcus mutans, and influenza.

41. The vaccine composition of claim 40, wherein administration of the double mutant Bordetella bacteria immunizes the mammal against a disease caused by the pathogen from which the antigen was derived.

42. A live attenuated vaccine composition for immunizing a mammal against a disease comprising an immunizing amount of a double mutant bacteria, wherein the bacteria is avirulent and induces an immune response upon administration to a mammal; and a pharmaceutically acceptable carrier.

43. A method of immunizing a mammal against a disease caused by a pathogen comprising:
administering to a susceptible mammal an immunizing amount of a double mutant Bordetella bacteria, wherein at least one gene of the Type III secretion system and at least one gene of the adenylate cyclase toxin (CyaA) locus of the bacteria each comprise at least one mutation so that the Bordetella bacteria produces no Type III secretion system or a non-functional Type III secretion system and no CyaA protein or a non-functional CyaA protein, wherein the double mutant Bordetella bacteria further comprises a heterologous gene encoding an antigen derived from the pathogen and a pharmaceutically acceptable carrier, thereby immunizing the mammal against the disease caused by the pathogen from which the antigen was derived.

44. The method of claim 43, wherein the heterologous gene that encodes the antigen derived from the pathogen selected from the group consisting of Leptospira canicola, Leptospira grippotyphosa, Leptospira hardjo, Leptospira ictero-haemorrhagiae, Leptospira pomona, Leptospira interrogans, Leptospira bratislava, canine distemper virus, canine adenovirus type 2, canine parainfluenza virus, canine parvovirus, rabies, herpes viruses, HIV, Erysipelothrix rhusiopathiae, Pasteurella, Pasteurella multocida, Ascaris, Oesophagostomum, pseudorabies virus, porcine parvovirus, pathogenic Escherichia coli, Bacillus anthracis, respiratory syncytial virus, Porcine Reproductive and Respiratory Syndrome Virus (PRRSV), swine influenza virus (SIV), porcine circovirus (PCV), Clostridium, Salmonella, Vibrio, Mycoplasma, Actinobacillus pleuropneumoniae, Haemophilus, rotavirus, transmissible gastroenteritis virus, Streptococcus sobrinus, Streptococcus mutans, and influenza.

45. A method for treating a disease caused by a pathogen in a mammal comprising: administering to a susceptible mammal an effective amount of a double mutant Bordetella bacteria, wherein at least one gene of the Type III secretion system and at least one gene of the adenylate cyclase toxin (CyaA) locus of the bacteria each comprise at least one mutation so that the Bordetella bacteria produces no Type III secretion system or a non-functional Type III secretion system and no CyaA protein or a non-functional CyaA protein, wherein the double mutant Bordetella bacteria further comprises a heterologous gene encoding an antigen derived from the pathogen and a pharmaceutically acceptable carrier, thereby treating in the mammal a disease caused by the pathogen from which the antigen was derived.

46. The method of claim 45, wherein the double mutant Bordetella bacteria further comprises a heterologous gene that encodes an antigen derived from a pathogen selected from the group consisting of Leptospira canicola, Leptospira grippotyphosa, Leptospira hardjo, Leptospira ictero-haemorrhagiae, Leptospira pomona, Leptospira interrogans, Leptospira bratislava, canine distemper virus, canine adenovirus type 2, canine parainfluenza virus, canine parvovirus, rabies, herpes viruses, HIV, Erysipelothrix rhusiopathiae, Pasteurella, Pasteurella multocida, Ascaris, Oesophagostomum, pseudorabies virus, porcine parvovirus, pathogenic Escherichia coli, Bacillus anthracis, respiratory syncytial virus, Porcine Reproductive and Respiratory Syndrome Virus (PRRSV), swine influenza virus (SIV), porcine circovirus (PCV), Clostridium, Salmonella, Vibrio, Mycoplasma, Actinobacillus pleuropneumoniae, Haemophilus, rotavirus, transmissible gastroenteritis virus, Streptococcus sobrinus, Streptococcus mutans, and influenza.
